(19) United States
(12) Patent Application Publication
(10) Pub. No.: US 2021/0113191 A1
(43) Pub. Date: Apr. 22, 2021

(54) IMAGE DATA ADJUSTMENT METHOD AND DEVICE

(71) Applicant: SHENZHEN MINDRAY BIO-MEDICAL ELECTRONICS CO., LTD., Shenzhen, Guangdong (CN)

(72) Inventors: Yaoxian ZOU, SHENZHEN (CN); Muqing LIN, SHENZHEN (CN); Gang ZHAO, SHENZHEN (CN); Tao JIN, SHENZHEN (CN)

(21) Appl. No.: 16/608,584

(22) PCT Filed: Apr. 26, 2017
(86) PCT No.: PCT/CN2017/082033

§ 371 Date: May 8, 2020

(51) Int. Cl. A61B 8/08; A61B 8/00; A61B 8/14

(52) U.S. Cl. CPC A61B 8/483; A61B 8/466; A61B 8/14; A61B 8/467; A61B 8/42; A61B 8/565

(57) ABSTRACT

An image data adjustment method comprises: acquiring three-dimensional ultrasound volume data of a detected target organism; extracting first section image data, at a first position, in the three-dimensional ultrasound volume data; when an adjustment instruction output by an adjustment portion is acquired, acquiring a prediction path corresponding to the first section image data; adjusting the first position in the three-dimensional ultrasound volume data to a second position along the prediction path; and acquiring second section image data, at the second position, in the three-dimensional ultrasound volume data, and displaying the second section image data.

### TECHNICAL FIELD

The present disclosure relates to computer technologies, particularly to an image data adjustment method and device.

### BACKGROUND

With the development and advancement of technologies, various medical devices have been widely used in clinical medicine. For example, ultrasound devices, as the main auxiliary equipments for clinical medical treatment, can scan the tissue or organ to be examined and output the three-dimensional image of the scanned objects, thereby helping the doctors to make correct judgments about the health of the body. In the prior art, after the three-dimensional ultrasound device scans the body, the three-dimensional image can be adjusted by conventional three-dimensional operations to obtain a specific section image in the three-dimensional ultrasound volume data. For example, after obtaining the three-dimensional ultrasound volume data of the head by scanning, the three-dimensional ultrasound device can display the specific section image of the three-dimensional ultrasound volume data, such as the cerebellum section image or the lateral ventricle section image, etc. However, the conventional three-dimensional operations include a variety of adjustment operations, and multiple adjustment attempts by multiple knobs are required to obtain a better section image. The complexity of obtaining the specific section image in the three-dimensional ultrasound volume data is increased.

### SUMMARY

The embodiments of the present disclosure provide image data adjustment methods and devices, which can automatically find out a desired specific section image according to user requirements, and can reduce the complexity of obtaining a specific section image in a three-dimensional ultrasound volume data.

Therefore, in one embodiment of the present disclosure, an image data adjustment method is provided, which may include:
obtaining a three-dimensional ultrasound volume data of an examined target body;
determining a prediction mode for adjusting an orientation of a section in the three-dimensional ultrasound volume data;
obtaining an image data from the three-dimensional ultrasound volume data according to the prediction mode; and
displaying the obtained image data.

In one embodiment, image data adjustment device is provided, which may include:
a volume data obtaining unit configured to obtain a three-dimensional ultrasound volume data of an examined target body;
a prediction adjustment unit configured to determine a prediction mode of adjusting an orientation of a section in the three-dimensional ultrasound volume data and obtain an image data from the three-dimensional ultrasound volume data according to the prediction mode; and
a display unit configured to display the obtained image data.

In one embodiment, an ultrasound imaging device is provided, which may include an ultrasound probe, a transmitting and receiving circuit, an image processing unit, a human-computer interaction device, a display, a memory and a processor.

The ultrasound probe may be configured to transmit ultrasonic waves to a target body.

The transmitting and the receiving circuit may be configured to excite the ultrasound probe to transmit an ultrasonic beam to the target body and receive echoes of the ultrasonic beam to obtain ultrasound echo signals.

The image processing unit may be configured to obtain a three-dimensional ultrasound volume data according to the ultrasound echo signals.

The human-computer interaction device may be configured to obtain an instruction inputted by an user.

The memory may be configured to store a computer program.

The processor may be configured to execute the computer program which, when executed by the processor, lead the processor to:
determine a prediction mode for adjusting an orientation of a section in the three-dimensional ultrasound volume data;
obtain an image data from the three-dimensional ultrasound volume data according to the prediction mode; and
display the obtained image data.

### BRIEF DESCRIPTION OF THE DRAWINGS

In order to more clearly illustrate the embodiments of the present disclosure or the technical solutions in the prior art, the drawings to be used in the description of the embodiments or the prior art will be briefly described below. Obviously, the drawings in the following description are only certain embodiments of the present disclosure, and other drawings can be obtained according to these drawings by those skilled in the art without any creative work.

FIG. 1 is a schematic flow chart of a three-dimensional imaging method in one embodiment of the present disclosure;

FIG. 2 is a schematic flow chart of an image data adjustment method in one embodiment of the present disclosure;

FIG. 3 is a schematic flow chart of another image data adjustment method in one embodiment of the present disclosure;

FIG. 4 schematically shows section images in one embodiment of the present disclosure;

FIG. 5a is a schematic diagram showing the position of an adjustment unit in one embodiment of the present disclosure;

FIG. 5b is a schematic diagram showing the position of another adjustment unit in one embodiment of the present disclosure;

FIG. 5c is a schematic diagram showing the position of another adjustment unit in one embodiment of the present disclosure;

FIG. 6 is a schematic flow chart of another image data adjustment method in one embodiment of the present disclosure;

FIG. 7a schematically shows another section image in one embodiment of the present disclosure;

FIG. 7b schematically shows another section image in one embodiment of the present disclosure;

FIG. 8 is a schematic flow chart of another image data adjustment method in one embodiment of the present disclosure;

FIG. 9 is a schematic diagram of a customized path in one embodiment of the present disclosure;

FIG. 10 is a schematic diagram of an operation interface in one embodiment of the present disclosure;

FIG. 11 is a schematic diagram of another operation interface in one embodiment of the present disclosure;

FIG. 12 is a schematic flowchart diagram of an image data adjustment method in one embodiment of the present disclosure;

FIG. 13 is a schematic structural diagram of an image data processing device in one embodiment of the present disclosure;

FIG. 14 is a schematic structural diagram of a prediction adjustment module in one embodiment of the present disclosure;

FIG. 15 is a schematic structural diagram of another image data processing device in one embodiment of the present disclosure;

FIG. 16 is a schematic structural diagram of a first data extraction unit in one embodiment of the present disclosure;

FIG. 17 is a schematic structural diagram of a prediction path obtaining unit in one embodiment of the present disclosure;

FIG. 18 is a schematic structural diagram of another image data processing device in one embodiment of the present disclosure;

FIG. 19 is a schematic structural diagram of another prediction adjustment module in one embodiment of the present disclosure; and

FIG. 20 is a schematic structural diagram of another image data processing device in one embodiment of the present disclosure.

### DETAILED DESCRIPTION

The technical solutions in the embodiments of the present disclosure will be clearly and completely described in the following with reference to the drawings. It will be obvious that the described embodiments are only a part, but not all, of the embodiments of the present disclosure. All other embodiments obtained by a person ordinarily skilled in the art based on the embodiments of the present disclosure without creative efforts will fall in the scope of the present disclosure.

In the embodiment of the present disclosure, by acquiring the three-dimensional ultrasound volume data of the detection target body, a prediction mode for adjusting the orientation of a section in the three-dimensional volume data may be determined, the image data may be extracted from the three-dimensional ultrasound volume data according to the prediction mode, and the section image may be displayed according to the extracted image data. Thereby, the position of the section in the three-dimensional ultrasound volume data can be adjusted according to the prediction mode, and the fine adjustment to the ultrasound section when using the three-dimensional ultrasound imaging device to examine the body tissue can be achieved. In the present embodiment, an input of the user may be used to activate a certain prediction adjustment mode. The prediction mode mentioned in this embodiment may include a fine adjustment mode in which the position of a specific section is finely adjusted based on the adjustment instruction input by the user, and a selection mode in which the user selects a specific section according to multiple sections provided by the system. The specific implementation of two prediction modes will be provided below.

For example, in one embodiment, an image data adjustment device (for example, an ultrasound device) may obtain a three-dimensional ultrasound volume data of a examined target body, and obtain the image data of a first section at a first position in the three-dimensional ultrasound volume data. When an adjustment instruction outputted by an adjustment unit is obtained, the image data adjustment device may obtain a prediction path, and adjust the section from the first position to a second position in the three-dimensional ultrasound volume data along the prediction path. Thereafter, the image data adjustment device may obtain a second section image data located at the second position in the three-dimensional ultrasound volume data, and display the second section image data to obtain a section image. By automatically obtaining the prediction path corresponding to the first image data and automatically adjusting the first section at the first position in the three-dimensional ultrasound volume data according to the prediction path to obtain the second section image data, the complexity of obtaining a specific section image in the three-dimensional ultrasound volume data is reduced.

For another example, in another embodiment, an image data adjustment device (for example, an ultrasound device) may obtain a three-dimensional ultrasound volume data of a examined target body, determine a spatial search path which may include at least two target positions, obtain at least two section image data from the three-dimensional ultrasound volume data along the spatial search path, and display the at least two section image data to obtain at least two section images for selection by an user. In this embodiment, the section images can be automatically obtained based on a certain path inputted by the user for the user to select which one is the most desired specific section image. It is convenient and quick, and can also enable the user to observe a plurality of section images near the anatomical structure.

The two prediction methods may also be freely switched based on the input of the user. For example, by identifying whether what is inputted by the user is a spatial search path or an adjustment instruction inputted through the adjustment unit, it can be determined which adjustment method in the two embodiments above will be used. The two methods can be switched freely, which is convenient and reliable. When the ultrasound image is displayed and operated by a touch screen, it will be more convenient and quicker, and have better experience. In the former prediction method, how to adjust the orientation of the section in the three-dimensional volume data may be predicted according to the prediction path corresponding to the section. For example, the prediction path corresponding to a section in a certain orientation may be obtained according to the priori data, thereby providing a predicted trajectory for the adjustment of such section. In the latter prediction method, the section image data at at least two adjacent positions may be automatically obtained according to the spatial search path, such that the user can select a satisfactory specific section image therefrom.

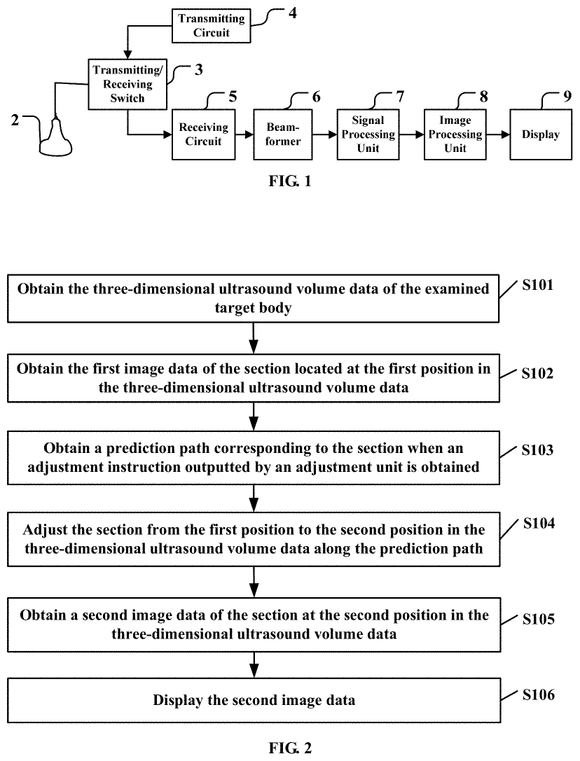

The image data adjustment devices in the embodiments of the present disclosure may be ultrasound imaging devices having a three-dimensional ultrasound imaging system. As shown in FIG. 1, the three-dimensional ultrasound imaging system may include a probe, a transmitting/receiving switch, a transmitting circuit, a receiving circuit, a beam-former, a signal processing unit, an image processing unit and a display. In the ultrasound imaging process, the transmitting circuit 4 may transmit delayed-focused transmitting pulses having a certain amplitudes and polarities to the ultrasound probe 2 through the transmitting/receiving switch 3. The ultrasound probe 2 may be excited by the transmitting pulses to transmit ultrasound waves (which may be any one of plane waves, focused waves and divergent waves) to a target body (for example, a certain tissue in the human body or an animal body and the vessel thereof, etc., not shown). After a certain delay, the ultrasound probe 2 may receive the ultrasound echoes with the information of the target body returning from the target area and convert the ultrasound echoes into electrical signals. The receiving circuit 5 may receive the electrical signals generated by the ultrasound probe 2 to obtain ultrasound echo signals and send the ultrasound echo signals to the beam-former 6. The beam-former 6 may perform processing such as focus delay, weighting, and channel summation, etc. on the ultrasound echo signals, and then send the ultrasound echo signals to the signal processing unit 7 where related signal processing may be performed thereon. The ultrasound echo signals processed by the signal processing unit 7 may be sent to the image processing unit 8. The image processing unit 8 may perform different processing on the signals according to different imaging modes desired by the user to obtain ultrasound image data of different modes, and perform processing such as logarithmic compression, dynamic range adjustment and digital scan conversion, etc. on the ultrasound image data to obtain ultrasound images of different modes such as B image, C image or D image, etc., or obtain a three-dimensional ultrasound image. The ultrasound image data may be displayed by the display 9, such as displaying a two-dimensional section ultrasound image or a three-dimensional ultrasound image. The three-dimensional ultrasound image may be obtained by scanning with a 2D matrix probe. Alternatively, the three-dimensional ultrasound image may be obtained by reconstructing a series of two-dimensional ultrasound image data obtained by scanning with a 1D linear probe. In some embodiments of the present disclosure, the signal processing unit and the image processing unit in FIG. 1 may be integrated on one motherboard. Alternatively, one or two or more of the units may be implemented in one or more processors/controller chips.

An image data adjustment method in one embodiment of the present disclosure will be described in detail below with reference to FIG. 2 to FIG. 9.

FIG. 1 is a schematic flow chart of an image data adjustment method in one embodiment of the present disclosure. As shown in FIG. 2, the method in the embodiment of the present disclosure may include the following steps S101 to S106.

In step S101, a three-dimensional ultrasound volume data of a target body may be obtained.

In some embodiments of the present disclosure, specifically, a processor in the image data adjustment device may obtain the three-dimensional ultrasound volume data of the examined target body. It may be understood that the target body may be a tissue or an organ of a human or an animal, such as brain tissue or cardiovascular tissue, etc. The three-dimensional ultrasound volume data above may be an ultrasound volume data obtained by scanning the target body with the ultrasound probe in the image data adjustment device and processing the data with the processor, such as an intracranial three-dimensional ultrasound volume data obtained by scanning the brain tissue. Alternatively, the three-dimensional ultrasound volume data above may be a three-dimensional ultrasound volume data obtained from another three-dimensional ultrasound imaging system or server through a network. The three-dimensional ultrasound volume data here may be obtained by direct scanning using a 2D matrix probe, or be obtained by reconstructing a series of two-dimensional ultrasound image data obtained by scanning with 1D probe.

In step S102, a first image data of a section located at a first position in the three-dimensional ultrasound volume data may be obtained;

In some embodiments of the present disclosure, specifically, the processor may obtain the first image data of the section located at the first position in the three-dimensional ultrasound volume data. It can be understood that the first position may be a display position of the first image data in the three-dimensional ultrasound volume data when the image data adjustment device obtain the three-dimensional ultrasound volume data by scanning. The first image data of the section may be an image data representing a specific cross section of the body tissue related to a human or animal body anatomical orientation in the three-dimensional ultrasound volume data. For example, the first image data of the section may be the image data of the cerebellar section in an intracranial three-dimensional ultrasound volume data obtained by scanning a fetal brain tissue. The first image data of the section may include the image data of at least one section. In some embodiments of the present disclosure, the section may be a section corresponding to any one orientation in the three-dimensional ultrasound volume data. For example, taking the image of the brain or cardiac tissue of a fetus as an example, the section may be any one of a cerebellar section, a thalamic section, a lateral ventricle section, a median sagittal section, a four-chamber heart section, a left ventricular outflow tract section, a right ventricular outflow tract section, a three-vessel tracheal section, a gastric vesicle section and an arterial catheter arch section, etc., and a combination thereof.

In some embodiments of the present disclosure, the processor may automatically obtain the first image data of the section located at the first position from the three-dimensional ultrasound volume data. The automatic obtaining may be implemented with an automatic calculation of the calculation program, in which a certain section image data may be obtained using an automatic segmentation algorithm of the image. For example, the first image data of the median sagittal section may be automatically obtained from the ultrasound image according to the image characteristics based on the spatial orientation of the brain and the characteristics of the brain tissue.

In step S103, a prediction path corresponding to the section may be obtained when an adjustment instruction outputted by an adjustment unit is obtained.

It should be noted that, based on long-term clinical experience, each specific section image in a three-dimensional ultrasound volume data of the body tissue may correspond to an most or more frequently used adjustment mode, that is, to a most or more likely adjustment path (which is referred to as prediction path herein). The specific section may be a diagnostic section commonly used by doctors, or the section indicated in a standard medical examination procedure. The prediction path may be a transformation including one of a translation in the X, Y, and Z direction and a rotation in the X, Y, and Z directions or a combination thereof. For example, regarding the four-chamber heart section, the three-vessel tracheal section or the gastric vesicle section, etc. which are transverse section, the prediction path may be a translation in the Z direction; regarding the left ventricular outflow tract section, the prediction path may be a rotation in the Y direction; regarding the right ventricular outflow tract section and the arterial catheter arch section, the prediction path may be a rotation in the Z direction; regarding the median sagittal section, the prediction path may be a translation in the Y direction; and so on. Besides indicating which operation will be performed in which direction, the prediction path may also include the step size of the operation performed in the direction. For example, regarding the left ventricular outflow tract section, the prediction path may be a rotation in the Y direction by 1 degree; regarding the right ventricular outflow tract section and the arterial catheter arch section, the prediction path may be a rotation in the Z direction by 2 degrees; regarding the median sagittal section, the prediction path may be a translation in the Y direction by 2 units; and so on. It can be understood that the prediction path may include a combination of at least one of the moving direction and the operation mode and a moving range (the moving range may include a distance and/or an angle). The prediction path corresponding to each specific section may be stored in the image data adjustment device, that is, the prediction path corresponding to each section image data may be known. In the embodiments of the present disclosure, it will not be limited to the specific section, but may be applied to the adjustment for any section. That is to say, the prediction path corresponding to each section orientation may be stored in the image data adjustment device.

It can be understood that, when the orientation of the section in the three-dimensional ultrasound volume data is different, the prediction path corresponding to the section will be different. The section image desired to be observed in the three-dimensional ultrasound volume data may not be determined in one time. For example, the first position corresponding to the four-chamber heart section automatically obtained from the three-dimensional ultrasound volume data of the heart may be offset to the left or right relative to the desired position thereof. If the four-chamber heart section is to be adjusted to a desired position suitable for observation (for example, a central position in the three-dimensional ultrasound volume data of the heart chamber), an auxiliary manual operation may be desired to participate in the adjustment. When the four-chamber heart section is offset to the left, the corresponding prediction path may be a translation to the right. When the four-chamber heart section is offset to the right, the corresponding prediction path may be a translation to the left. Usually, six knobs or buttons are used in the ultrasound system to perform manual adjustment of the section. The six knobs or buttons are X-axis translation, X-axis rotation, Y-axis translation, Y-axis rotation, Z-axis translation and Z-axis rotation. Therefore, it will be desired that the user has a very clear understanding to the difference between the image space and the physical space, and uses a combination operation of the six knobs or buttons to obtain the desired section image. This is very complicated and requires a good understanding to medical anatomy. Moreover, it is also necessary to be very familiar with the correspondence between the spatial orientation of the sections and the anatomical structures. Therefore, the difficulty and complexity of the use of the ultrasound device are increased. Based on this issue, in the present embodiment, the automatic obtainment or configuration of the prediction path may be activated according to the adjustment instruction inputted by the user through the adjustment unit to obtain the prediction path corresponding to the section, thereby reducing the number of buttons, decreasing the complexity of the operation, increasing the intelligence of the machine, reducing the cost of hardware, and achieving more miniaturization.

Further, when the orientation of the section in the three-dimensional ultrasound volume data is different, even the prediction path obtained according to the adjustment instruction inputted by the same adjustment unit will be different, because the prediction path corresponding to the adjustment unit will be automatically configured for different section. For example, in the case that the section is a four-chamber heart section, the prediction path based on the input inputted by a virtual button on the interface will be a translation in the Z-direction, while in the case that the section is the left ventricular outflow tract section, the prediction path based on the same input inputted by the virtual button will be a rotation in the Y-direction.

It can be understood that the adjustment instruction may be a control instruction which is inputted by the medical personnel through the adjustment unit of the image data adjustment device and triggers the three-dimensional ultrasound volume data. The adjustment unit may be a virtual adjustment unit or a physical adjustment unit. The virtual adjustment unit may include any graphic controls arranged on the display interface, such as any one of a key, a button and a slide bar arranged on the display interface of the section image data. The physical adjustment unit may be a hardware having a substantial shape, such as any one of physical hardware buttons, keys, knobs, scroll wheels and mouse.

Specifically, when obtaining the adjustment instruction inputted through the human-computer interaction device (that is, the adjustment unit), the processor may obtain a prediction path. For example, when the image data adjustment device performs a three-dimensional ultrasound examination on a human heart, the processor may obtain a prediction path of a section (four-chamber heart section) in the Z direction in the obtained three-dimensional ultrasound volume data.

In step S104, the section may be adjusted from the first position to the second position in the three-dimensional ultrasound volume data along the prediction path.

Specifically, the processor may adjust the section from the first position to the second position in the three-dimensional ultrasound volume data along the prediction path. It can be understood that the second position may be a display position of the section in the three-dimensional ultrasound volume data after the section is adjusted from the first position in the three-dimensional ultrasound volume data according to the prediction path. It can be understood that the processor may adjust the four-chamber heart section from the first position to the second position in the three-dimensional ultrasound volume data of the heart according to the translation in the Z-direction corresponding to the image data of the section (four-chamber heart section). In one embodiment, the prediction path may include any one of a prediction path of moving a preset distance in one direction and a prediction path of moving preset distances in at least two directions. When inputting the adjustment instruction through the adjustment unit, each time the adjustment instruction is input, the corresponding prediction path may be moving a preset distance in one direction or moving preset distances in at least two directions. The preset distance here may be measured in angle and/or displacement.

In step S105, a second image data of the section at the second position may be obtained from the three-dimensional ultrasound volume data.

Optionally, in the process of adjusting the section at the first position in the three-dimensional ultrasound volume data according to the prediction path corresponding to the first image data, the image data adjustment device may display in real time the image data of the section in the three-dimensional ultrasound volume data during the adjustment on the display screen. Optionally, the image data adjustment device may not display the adjustment process of the section, but directly display the section at the final position reached when the adjustment is completed, that is, at the second position. When the adjustment is completed and the section reaches the final position (that is, the second position), the image data adjustment device may display the image data of the section at the second position in the three-dimensional ultrasound volume data on the display screen, that is, display the second image data of the section.

Specifically, when the adjustment is completed, the processor may obtain the second image data of the section located at the second position in the three-dimensional ultrasound volume data. It can be understood that the second image data may be an image data in the cross section at the second position corresponding to the section at the first position. For example, the first image data at the first position is an image data of the four-chamber heart section, and the second image data at the second position is an image data of the four-chamber heart section translated in the Z-direction.

In S106, the second image data may be displayed to obtain the section image.

Specifically, the image data adjustment device may display the content of the second image data on the current display screen, such as displaying the image data of the four-chamber heart section translated in the Z direction. The image data of the section obtained in step 106 may not necessarily be the final desired image data of the section, and but may be an image data during the process of obtaining the desired image data of the section. That is, in the embodiment of the present disclosure, the second position corresponding to the desired image data of the section may be obtained by one input through the adjustment unit, or be obtained through adjustments to multiple second positions based on multiple inputs through the adjustment unit. Therefore, the prediction path in this embodiment will not be limited to adjust the section to the second position corresponding to the desired image data by one adjustment. Rather, in the prediction path in this embodiment, it may also be possible that the section is adjusted from the first position to the second position corresponding to the desired image data through step adjustments for gradually approaching the second position. The step adjustments may be performed according to the prediction direction and/or operation obtained by prior knowledge, thereby saving the adjustment time and reducing the adjustment complexity.

In the embodiments of the present disclosure, the three-dimensional ultrasound volume data of the detection target body may be obtained, and the first image data of the section at the first position may be obtained in the three-dimensional ultrasound volume data. When the adjustment instruction outputted by the adjustment unit is acquired, the prediction path corresponding to the first image data may be obtained, and the section may be adjusted from the first position to the second position in the three-dimensional ultrasound volume data along the prediction path. Thereafter, the second image data of the section at the second position in the three-dimensional ultrasound volume data may be obtained and displayed. By automatically obtaining the prediction path corresponding to the first image data and automatically adjusting the section at the first position in the three-dimensional ultrasound volume data according to the prediction path to obtain the second section image data, the complexity of obtaining a specific section image in the three-dimensional ultrasound volume data is reduced.

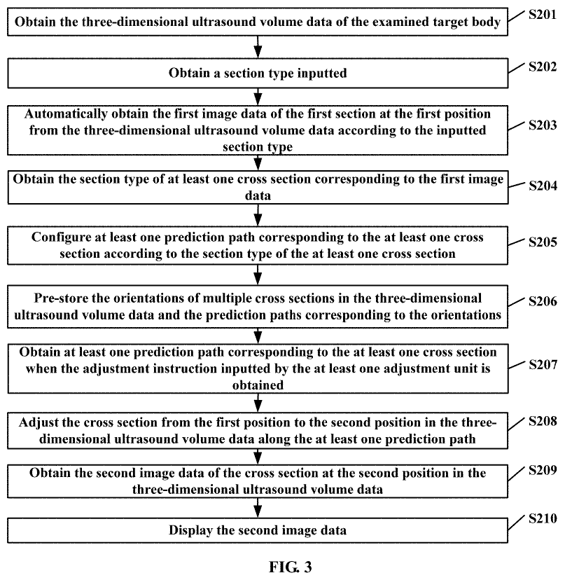

FIG. 3 is a schematic flowchart of an image data adjustment method in one embodiment of the present disclosure. As shown in FIG. 3, the method of the present embodiment may include the following steps S201 to S210.

In step S201, a three-dimensional ultrasound volume data of a target body may be obtained.

Specifically, the processor in the image data adjustment device may obtain the three-dimensional ultrasound volume data of the target body. It may be understood that the target body may be a tissue or an organ of a human or an animal, such as brain tissue or cardiovascular tissue, etc. The three-dimensional ultrasound volume data above may be a three-dimensional ultrasound volume data obtained by scanning the target body with the ultrasound probe in the image data adjustment device and processing the data with the processor, such as an intracranial three-dimensional ultrasound volume data obtained by scanning the brain tissue.

In step S202, a section type inputted may be obtained.

Specifically, the human-computer interaction device of the image data adjustment device may obtain the inputted section type. It can be understood that the section type may be a type name or a type number that represents the type of the section image data. For example, a type name of “four-chamber heart section” or a type number of “01” which is pre-agreed to represent the four-chamber heart section inputted by voice may be obtained. Each section type may correspond to a doctor's diagnostic cross section or a medical standard cross section, such as the four-chamber heart section, the three-vessel organ section, the gastric vesicle section, the median sagittal section of head, etc. In fact, different sections may correspond to different section orientations. Therefore, in fact, the section type is a specific form of the orientation of the section. The orientation of the section may be represented with the section type or the coordinates of the cross section in the three-dimensional ultrasound volume data.

In step S203, a first image data of the first section at the first position may be automatically obtained from the three-dimensional ultrasound volume data according to the inputted section type.

Specifically, the processor may automatically obtain the first image data at the first position from the three-dimensional ultrasound volume data according to the section type. It can be understood that the first position may be a display position of the first image data in the three-dimensional ultrasound volume data when the image data adjustment device obtain the three-dimensional ultrasound volume data by scanning. The first image data may be an image data representing a cross section of the body tissue related to a human or animal body anatomical orientation in the three-dimensional ultrasound volume data. For example, the first image data of the section may be the image data of the cerebellar section in an intracranial three-dimensional ultrasound volume data obtained by scanning a brain tissue.

In step S204, a section type of at least one cross section corresponding to the first image data may be obtained. When the first image data includes a plurality of cross sections, the section type of each cross section may be correspondingly determined according to the inputted section type.

It can be understood that the first image data may include at least one cross section. For example, the first image data in the intracranial three-dimensional ultrasound volume data may include the cerebellar section, the thalamic section or the lateral ventricle section, etc. It should be noted that the sections of different types may have different orientations in the three-dimensional volume data. For example, the cerebellar section may be in an upper orientation in the intracranial three-dimensional ultrasound volume data, while the thalamic section may be in a lower orientation in the intracranial three-dimensional ultrasound volume data.

Specifically, the processor may determine the section type of the cross sections in the at least one cross section corresponding to the first image data according to the image data of each cross section. For example, in the cast that the image data of the cross section indicates that it is an image of the cerebellum, it may be determined that section type of the cross section is cerebellar section.

In step S205, at least one prediction path corresponding to the at least one cross section may be configured according to the section type of the at least one cross section.

Specifically, the processor may arrange at least one prediction path corresponding to the at least one cross section according to the section type of the at least one cross section. It can be understood that the processor may arrange the prediction paths corresponding to the cross sections according to the section type of the cross sections of the at least one cross section. For example, in the case that the section type of a certain cross section in the at least one cross section is four-chamber heart section, the processor may arrange the prediction path of Z-direction translation for such cross section according to long-term clinical experience. It will be appreciated that each cross section in the at least one cross section may correspond to a prediction path obtained based on long-term clinical experience that is most frequently used.

In step S206, the orientations of a plurality of cross sections in the three-dimensional ultrasound volume data and the prediction paths corresponding to the orientations may be pre-stored.

Specifically, the orientation of the plurality of cross sections in the three-dimensional ultrasound volume data and the prediction paths corresponding to the orientation may be pre-stored in the memory of the image data adjustment device. For example, the image data adjustment device may pre-store the upper middle orientation of the median sagittal section in the three-dimensional volume data and the prediction path of translation in the negative Y-direction corresponding to such orientation.

It can be understood that the different orientations of the cross section image data in the three-dimensional ultrasound volume data may correspond to different section types. The prediction path may be directly searched according to the value representing the orientation, or according to the section type. For different orientations of the first image data in the three-dimensional ultrasound volume data, the prediction paths corresponding to the first image data will be different. For example, due to the difference in acquiring the three-dimensional ultrasound volume data, the orientation of the obtained first image data in the three-dimensional ultrasound volume data is different (for example, the position of the four-chamber heart section in the three-dimensional ultrasound volume data of the heart may be on the left or right). When it is desired to adjust the four-chamber heart section to a position suitable for observation (for example, the middle position in the three-dimensional ultrasound data of the heart), the prediction path will be translation to the right when the four-chamber heart section is on the left and be translation to the left when the four-chamber heart section is on the right.

Further, for different orientations of the first image data in the three-dimensional ultrasound volume data, even the prediction paths obtained according to the adjustment instruction inputted by the same adjustment unit may be different. For example, when the first image data is a four-chamber section image data, the prediction path obtained according to the input of the virtual button on the interface may be a translation in the Z-direction, while, when the first image data is a left ventricular outflow tract section image data, the prediction path obtained according to the input of the same virtual button may be a rotation in the Y-direction.

In the embodiment of the present disclosure, by storing the plurality of section image data and corresponding prediction paths in advance, the accuracy of automatically obtaining the prediction path according to the section image can be increased.

In step S207, at least one prediction path corresponding to the at least one cross section may be obtained when obtaining an adjustment instruction inputted by the at least one adjustment unit.

It can be understood that the number of the adjustment units in the image data adjustment device may be the same as the number of the cross sections currently displayed. That is, in the case that the image data adjustment device has four adjustment units, the display screen of the image data adjustment device may be divided into four regions to display the image data of four cross sections. It can be understood that the processor may obtain at least one prediction path corresponding to the at least one cross sections according to the adjustment instruction inputted by at least one adjustment unit, and may accordingly perform adjustment on the three-dimensional ultrasound volume data according to the prediction path corresponding to each cross section.

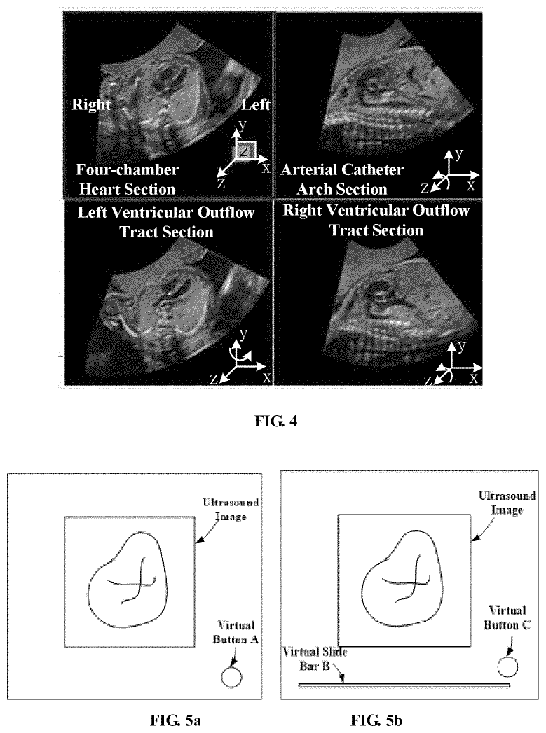

Specifically, when obtaining the adjustment instruction inputted by the user through the human-computer interaction device (that is, the at least one adjustment unit), the processor may obtain at least one prediction path corresponding to the at least one cross section. As shown in FIG. 4, there are 4 cross sections (the four-chamber heart section, the arterial catheter arch section, the left ventricular outflow tract section and the right ventricular outflow tract section) on the display, and the prediction paths corresponding to the cross sections are Z-direction translation, Z-direction rotation, Y-direction rotation and Z-direction rotation, respectively.

In a specific implementation of one embodiment of the present disclosure, the image data adjustment device may have one or more adjustment units.

Optionally, in the case that the image data adjustment device has one adjustment unit (as shown in FIG. 5a, the virtual button A on the display screen of the image data adjustment device), the one adjustment unit may be used to perform any adjustment in direction, manner and distance. For example, with the virtual button A, a movement of preset distance in one direction may be achieved, in which the movement of preset distance may include a translation of preset distance and a rotation of preset angle (e.g., a translation of 1 mm in X direction or a rotation of 1 degree around X axis), or, a movement of preset distance in at least two directions may be achieved (e.g., a translation of 1 mm in X direction and a translation of 1 mm in Y direction).

Optionally, in the case that the image data adjustment device has two adjustment units (as shown in FIG. 5b, the virtual sliding bar B and the virtual button C on the display screen of the image data adjustment device), the two adjustment units may correspond to two adjusting manners. For example, the virtual sliding bar B may be used to perform the translation adjustment in the X, Y and Z directions, and the virtual button C may be used to perform the rotation adjustment around the X, Y and Z directions.

Optionally, in the case that the image data adjustment device has three adjustment unit (as shown in FIG. 5c, the virtual button D, the virtual knob E and the virtual sliding bar F on the display screen of the image data adjustment device), the three adjustment units may respectively correspond to the adjustment in three directions. For example, adjusting the virtual button D may achieve a movement of preset distance in the X direction, adjusting the virtual knob E may achieve a movement of preset distance in the Y direction, and adjusting the virtual sliding bar F may achieve a movement of preset distance in the Z direction.

Regardless of the number of the adjustment units, reconfiguring the preset distance of the movement corresponding to the adjustment instruction outputted by the adjustment unit according to the prediction path obtained through the first image data may also be understood as reconfiguring the adjustment operation mode and the adjustment step size, where the adjustment step size may be angle or displacement. Each time a different cross section is selected for adjustment, the reconfiguring of the prediction path may be performed for the adjustment unit.

In step S208, the cross section may be adjusted from the first position to a second position in the three-dimensional ultrasound volume data along the at least one prediction path.

Specifically, the processor may adjust the cross section from the first position to the second position in the three-dimensional ultrasound volume data along the at least one prediction path. As shown in FIG. 4, the processor may adjust the cross section from the first position to the second position in the three-dimensional ultrasound volume data simultaneously according to the Z-direction translation corresponding to the four-chamber heart section, the Z-direction rotation corresponding to the arterial catheter arch section, the Y-direction rotation corresponding to the left ventricular outflow tract section and the Z-direction rotation corresponding to the right ventricular outflow tract section.

In the embodiments of the present disclosure, the prediction path may usually be one of the six basic adjustment modes of rotation and translation in the X, Y and Z directions. That is, the dimensionality reduction method from 6-dimensional space to 1-dimensional space may be used, in which one certain dimension of the six dimensions is directly taken according to the orientation of the cross section in the human anatomy. In other embodiments, the dimensionality reduction method may also be a linear or non-linear combination of the 6-dimensional parameters, such as a combination of translations in X and Y direction in which the translations in X and Y direction may be achieved simultaneously when the corresponding adjustment unit is adjusted. The dimensionality reduction may also be implemented according to the anatomical features of the cross section using a machine learning method. For example, the user's usual operating habits may be recorded by the machine and stored as data, and the most common operation path of the user may be obtained therefrom by the machine learning algorithm. Such most common operation path may be the most likely prediction path. The commonly used machine learning algorithms may be support vector machine (SVM), principal component analysis (PCA), convolutional neural network (CNN), recurrent neural network (RNN) and the like.

It can be understood that the processor may adjust the cross section at the first position in the three-dimensional ultrasound volume data using the prediction path obtained according to any one of the six dimensional spatial parameters, the linear or non-linear combination of the six dimensional spatial parameters or the conventional adjustment path obtained by the machine learning, etc.

In step S209, the second image data of the cross section at the second position in the three-dimensional ultrasound volume data may be obtained.

Optionally, during the movement of the cross section in the first position in the three-dimensional ultrasound volume data according to the prediction path corresponding to the first image data, the change of the first image data in the three-dimensional ultrasound volume data may be displayed on the display screen of the image data adjustment device in real time. Optionally, the display screen of the image data adjustment device may also not display the adjustment process of the cross section at the first position, but directly display the image data at the final position reached when the adjustment is completed, that is, at the second position. When the adjustment is completed and the cross section reaches the final position, that is, the second position, the display screen may display the state of the cross section at the second position in the three-dimensional ultrasound volume data, that is, display the second image data of the cross section.

Specifically, when the adjustment is completed, the processor may obtain the second image data of the cross section at the second position in the three-dimensional ultrasound volume data. It can be understood that the second image data may be an image data of the cross section corresponding to the first image data at the second position. For example, the first image data at the first position is the image data of the four-chamber heart section, and the second image data at the second position is the image data of the four-chamber heart section translated in the Z direction. Further, in the case that the first image data corresponds to at least one cross section, the second image data may also correspond to at least one cross section.

In step S210, the second image data may be displayed to obtain the section image.

Specifically, the image data adjustment device may display the image data content of the second image data in the current display screen. For example, the image data of the four-chamber heart section translated in the Z direction, the image data of the arterial catheter arch section rotated in the Z direction, the image data of the left ventricular outflow tract section rotated in the Y direction and the image data of the right ventricular outflow tract section rotated in the Z direction may be simultaneously displayed.

In the embodiments of the present disclosure, the section at the first position in the three-dimensional ultrasound volume data may be adjusted according to at least one prediction path corresponding to the at least one cross section corresponding to the first image data. Therefore, the diversity of the adjustment to the cross section in the three-dimensional ultrasound volume data may be increased.

In the embodiments of the present disclosure, the three-dimensional ultrasound volume data of the target body may be obtained, and the first image data of the section at the first position may be obtained from the three-dimensional ultrasound volume data. When the adjustment instruction output by the adjustment unit is acquired, the prediction path may be obtained, and the section at the first position may be adjusted to the second position in the three-dimensional ultrasound volume data along the prediction path. Thereafter, the second image data of the section at the second position in the three-dimensional ultrasound volume data may be obtained, and displayed. By automatically obtaining the prediction path corresponding to the first image data and automatically adjusting the section at the first position in the three-dimensional ultrasound volume data according to the prediction path to obtain the second image data of the section, the complexity of adjusting the cross section in the three-dimensional ultrasound volume data may be reduced. By pre-storing the plurality of section image data and their corresponding prediction paths, the accuracy of automatically obtaining the prediction path according to the section image may be increased. By adjusting the section at the first position in the three-dimensional ultrasound volume data according to at least one prediction path corresponding to the at least one cross section corresponding to the first image data, the diversity of the adjustment to the cross section in the three-dimensional ultrasound volume data may be increased.

The type of the section mentioned above may also be a specific form of the orientation of the section. Therefore, in the embodiments of the present disclosure, it will not be limited to configure or find the prediction path only according to the type of the section. In some embodiments, the first image data of the section at the first position may be automatically obtained from the three-dimensional ultrasound volume data according to the orientation of the section inputted by the user. In addition, the prediction path may be searched and the adjustment unit may be reconfigured according to the orientation of the section.

In one embodiment, before the step 104 in FIG. 2, the method may further include the following steps:
searching for the prediction path corresponding to the first image data, such as searching for the prediction path according to the orientation of the first image data in the three-dimensional ultrasound volume data; and
associating the adjustment instruction outputted by the adjustment unit and the searched prediction path to reconfigure the adjustment unit according to the searched prediction path. Each time the orientation of the first image data is changed, the prediction path may be associated by reconfiguring the adjustment unit, thereby reducing the complexity of each section adjustment and conveniently and quickly adjusting the section to the desired position.

Further, the first image data may include at least one section image data. Therefore, the correspondence between the adjustment instruction outputted by the adjustment unit and the prediction path may be reconfigured according to a selected one of the at least one section image data. When the adjustment instruction outputted by the adjustment unit is acquired, the reconfigured prediction path may be obtained, and the position of the selected section may be finely adjusted according to the reconfigured prediction path. In this way, the position of the section may be accurately positioned using as limited number of adjustment units as possible, thereby reducing the adjustment difficulty and facilitating user operation.

In a possible implementation of the embodiment of the present disclosure, obtaining the prediction path when obtaining the adjustment instruction outputted by the adjustment unit may include the following steps, as shown in FIG. 6.

In step S301, a current position of an indication identifier in the current screen may be obtained.

It can be understood that a plurality of section image data may be simultaneously displayed on the display screen of the image data adjustment device. When the plurality of image data are simultaneously displayed on the display screen, the processor in the image data adjustment device may perform the adjustment on one of the plurality of section image data.

Specifically, when multiple section image data are displayed on the display screen at the same time, the system may usually provide a way for activating one of the sections. When a certain section is activated, all subsequent operations may be performed on the activated section.

For example, the processor may obtain the current position of the indication identifier in the current screen. It may be understood that the indication identifier may be the cursor in the current screen, and the user may place the cursor on the position of the section image data to be adjusted of the multiple section image data displayed on the current screen to activate the section image data to be adjusted. Thereby, the processor can obtain the current position of the cursor. It can be understood that the current position of the indication identifier may be the position of the selected first image data, that is, the current position of the activated section.

In step S302, the first image data of the section at the current position may be obtained.

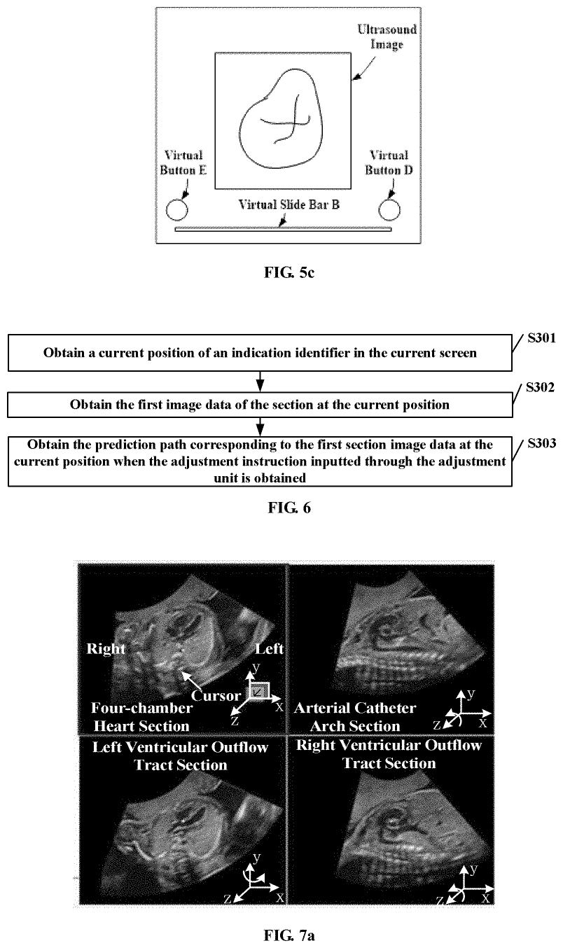

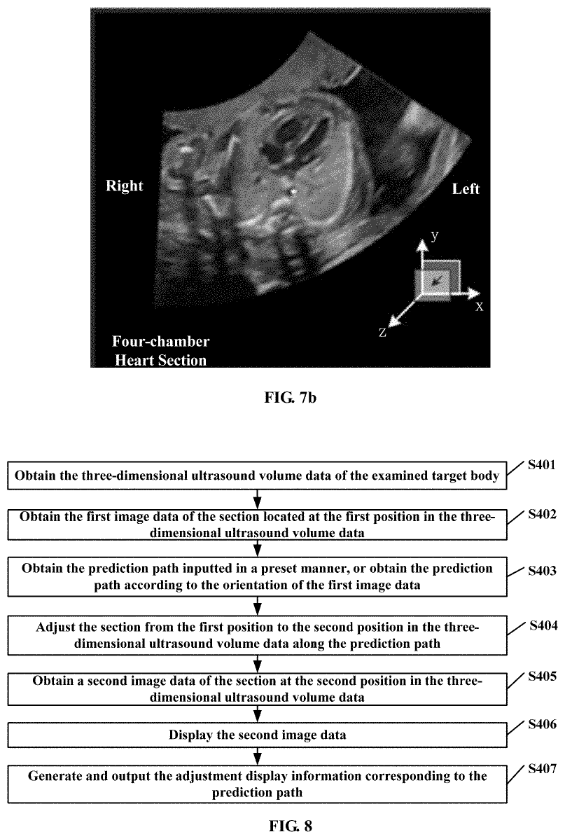

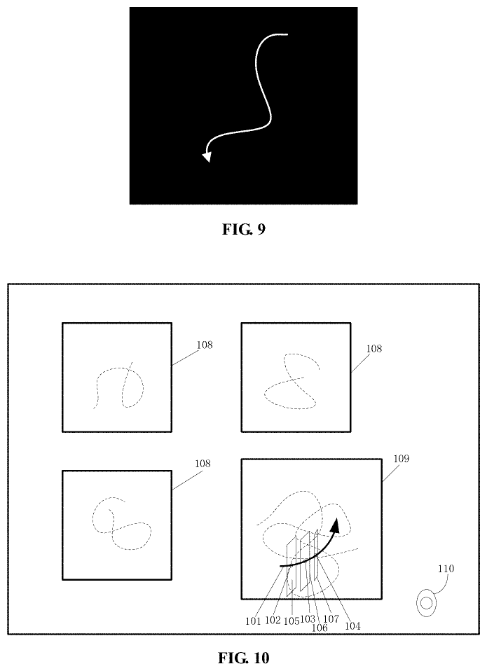

Specifically, the processor may obtain the first image data of the section at the current position. As shown in FIG. 7a, when the current position of the cursor is the position of a first section, such as the four-chamber heart section, the processor may obtain the first image data at such position, that is, the image data of the four-chamber heart section. Optionally, the processor may display only the currently selected first image data of the section through the display screen after selecting the first section image data, as shown in FIG. 7b.

In step S303, when obtaining the adjustment instruction inputted through the adjustment unit, the prediction path corresponding to the first section image data at the current position may be obtained.

Specifically, when the human-machine interaction module in the image data adjustment device (i.e. the adjustment unit) obtains the adjustment instruction input by the user through the adjustment unit, the processor may obtain the prediction path corresponding to the first image data at the current position. It can be understood that the position of the first image data in the three-dimensional ultrasound volume data and the corresponding prediction path have been stored in the image data adjustment device, and when the user activates the adjustment unit to adjust the section, the processor may directly retrieve the corresponding prediction path from the memory.

In the embodiments of the present disclosure, the first image data of the section may be selected by the cursor in the current screen, and the prediction path corresponding to the first image data may be obtained, thereby avoiding the adjustment to the section which needs not to be adjusted. Therefore, the unnecessary adjustment may be reduced, and the adjustment efficiency may be increased.

FIG. 8 is a schematic flowchart of an image data adjustment method according to one embodiment of the present disclosure. As shown in FIG. 8, the method of the present embodiment may include the following steps S401-S407.

In step S401, the three-dimensional ultrasound volume data of the target body may be obtained.

Specifically, the processor in the image data adjustment device may obtain the three-dimensional ultrasound volume data of the target body. It may be understood that the target body may be a tissue or an organ of a human or an animal, such as brain tissue or cardiovascular tissue, etc. The three-dimensional ultrasound volume data above may be an ultrasound volume data obtained by scanning the target body with the ultrasound probe in the image data adjustment device and processing the data with the processor, such as an intracranial three-dimensional ultrasound volume data obtained by scanning the brain tissue.

In step S402, the first image data of the section located at the first position in the three-dimensional ultrasound volume data may be obtained;

Specifically, the processor may obtain the first image data of the section located at the first position in the three-dimensional ultrasound volume data. It can be understood that the first position may be a display position of the first image data in the three-dimensional ultrasound volume data when the image data adjustment device obtain the three-dimensional ultrasound volume data by scanning. The first image data of the section may be an image data representing a specific cross section of the body tissue related to a human or animal body anatomical orientation in the three-dimensional ultrasound volume data. For example, the first image data of the section may be the image data of the cerebellar section in an intracranial three-dimensional ultrasound volume data obtained by scanning a fetal brain tissue.

In step S403, the prediction path inputted in a preset manner may be obtained; alternatively, the prediction path may be obtained according to the orientation of the first image data. Regarding the methods for obtaining the prediction path according to the orientation of the first image data, reference may be made to the related description of the embodiments above, which will not be described here again.

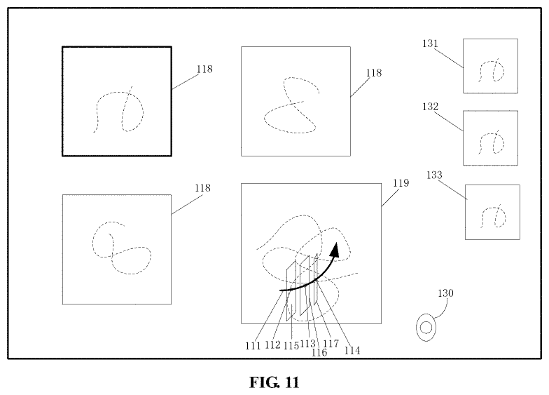

It can be understood that, in the embodiments of the present disclosure, the image data adjustment device may determine the prediction path with a user interactive method. For example, the user may draw a spatial search curve corresponding to a specific cross section of the fetal heart as shown in FIG. 9 in a certain manner. When the adjustment unit is activated, the orientation of the corresponding section may be adjusted along such curve, where the searched section may be orthogonal or tangent to the user-defined curve.

Specifically, the human-machine interaction module in the image data adjustment device may obtain the preset prediction path that is input by the user according to a preset manner. It can be understood that the preset manner may be a definition process of a spatial search curve implemented by an algorithm or a manner of manually drawing a spatial search curve by a screen cursor, such as the spatial search curve manually drawn by the cursor as shown in FIG. 9. The preset prediction path may be the custom spatial search curve.

In step S404, the section may be adjusted from the first position to the second position in the three-dimensional ultrasound volume data along the prediction path.

Specifically, the processor may adjust the section from the first position to the second position in the three-dimensional ultrasound volume data along the prediction path. For example, the section at the first position in the three-dimensional ultrasound volume data may be adjusted according to the spatial search curve corresponding to the specific cross section of the fetal heart as shown in FIG. 9.

In the embodiments of the present disclosure, the customized prediction path may be obtained, and the section at the first position in the three-dimensional ultrasound volume data may be adjusted according to the customized prediction path. Therefore, the accuracy of the adjustment can be increased.

In step S405, the second image data of the section at the second position may be obtained from the three-dimensional ultrasound volume data.

Optionally, in the process of adjusting the section at the first position in the three-dimensional ultrasound volume data according to the prediction path corresponding to the first image data, the image data adjustment device may display in real time the image data of the section in the three-dimensional ultrasound volume data during the adjustment on the display screen. Optionally, the image data adjustment device may not display the adjustment process of the section, but directly display the section at the final position reached when the adjustment is completed, that is, at the second position. When the adjustment is completed and the section reaches the final position (that is, the second position), the image data adjustment device may display the image data of the section at the second position in the three-dimensional ultrasound volume data on the display screen, that is, display the second image data of the section.

Specifically, when the adjustment is completed, the processor may obtain the second image data of the section at the second position in the three-dimensional ultrasound volume data. It can be understood that the second image data may be an image data in the cross section at the second position corresponding to the section at the first position. For example, the first image data at the first position is an image data of a specific cross section of the fetal heart, and the second image data at the second position is an image data of the specific cross section of the fetal heart moved along the spatial search curve shown in FIG. 9.

In step S406, the second image data may be displayed.

Specifically, the image data adjustment device may display the content of the second image data on the current display screen, such as displaying the image data of the specific cross section of the fetal heart moved along the spatial search curve shown in FIG. 9.

In step S407, adjustment display information corresponding to the prediction path may be generated and output.

It can be understood that, because the prediction paths of different cross sections are different, the image data adjustment device may generate the adjustment display information corresponding to the prediction paths so as to facilitate the understanding of the user. It can be understood that the adjustment display information may be a text, an icon, or other prompt information capable of informing the user the specific motion direction corresponding to the current prediction path. The adjustment display information may be the prompt information shown in FIG. 4, FIG. 7a and FIG. 7b, such as the prompt in FIG. 4, the indications in the x, y and z coordinate systems in FIG. 7a and FIG. 7b. Particularly, in the x, y and z coordinate systems of FIG. 7b, it is shown that two planes move in the direction indicated by the arrows, which represents the change of the position of the section when it is adjusted according to the prediction path.

Furthermore, the image data adjustment device may output the adjustment display information, such as display the prompt information shown in FIG. 4, FIG. 7a and FIG. 7b simultaneously with the second image data on the current display screen.

In the embodiments of the present disclosure, the specific movement directions during the adjustment may be displayed by the adjustment display information. Therefore, the degree of visualization of the adjustment process may be increased.

In the embodiments of the present disclosure, the three-dimensional ultrasound volume data of the examined target body may be obtained, and the first image data of the section at the first position may be obtained from the three-dimensional ultrasound volume data. Based on the preset prediction path inputted according to the preset manner, the section may be adjusted from the first position to the second position in the three-dimensional ultrasound volume data along the prediction path, and the second image data of the section at the second position in the three-dimensional ultrasound volume data may be obtained and displayed. In addition, the adjustment display information corresponding to the prediction path may be generated, and output. By obtaining the customized prediction path and adjusting the first section at the first position in the three-dimensional ultrasound volume data according to the customized prediction path, the accuracy of the adjustment is increased. By presenting the movement directions during the adjustment through the adjustment display information, the degree of visualization of the adjustment process is increased.

In addition, in the embodiments above, when the prediction path input in the preset manner is a spatial search path including at least two target positions, in one embodiment, as shown in FIG. 9, the spatial search path including the at least two target locations may be drawn in the two-dimensional section image or three-dimensional ultrasound image. The image data adjustment device may reconfigure the correspondence between the adjustment instructions output by the adjustment unit and the at least two target positions on the spatial search path. Thereafter, when the adjustment instruction output by the adjustment unit is obtained, the at least two target positions on the spatial search path may be searched, and at least two prediction paths may be obtained according to the at least two target positions. Thereafter, according to the input of the user through the adjustment unit, the first section at the first position may be gradually adjusted to the second position corresponding to the desired image data according to the prediction paths determined by the at least two target positions. In one embodiment, according to the obtained at least two prediction paths, the first section at the first position in the three-dimensional ultrasound volume data may be sequentially adjusted to multiple section positions along the at least two prediction paths until the position of the first section is moved to the desired position. In this process, each time the section is adjusted to a second position, the image data of the first section at such second position is obtained and displayed once, until the desired position is arrived and the desired section image data is obtained. The section image data at the multiple second positions in the three-dimensional ultrasound volume data may be tangent or orthogonal to the spatial search path. As shown in FIG. 10, three section images are displayed on the display interface, such as the images indicated by 108. In FIG. 10, the dashed lines indicate the area of the tissue being examined in the ultrasound image. A three-dimensional ultrasound image may be displayed generally in the area indicated by 109. In the present embodiment, a section image may also be displayed thereon. A spatial search path 101 (indicated by a black curve with arrow in the figure) is drawn in the area indicated by 109, and multiple target positions (102, 103, 104) on the spatial search path 101 may be determined and drawn, where the spatial search path 101 passes through the tissue being examined at the multiple target positions (102, 103, 104). The multiple target positions (102, 103, 104) may be determined on the spatial search path by a preset distance (e.g., in an equally spaced manner). Alternatively, specific anatomical structural points (e.g., mitral valve, right ventricular center point, etc.) in the tissue may be identified and the positions on or near the specific anatomical structural points and on the spatial search path may be determined as the target positions. Based on the determined target positions, the correspondence between the adjustment instruction output by the adjustment unit 110 and the at least two target positions on the spatial search path may be reconfigured. When the adjustment instruction output by the adjustment unit 110 is obtained, one target position may be obtained from the spatial search path, and thereby obtaining one corresponding prediction path from the first position to such target location. According to the input of the user through the adjustment unit 110, the first section at the first position corresponding to the current image data (the image in one of areas 108) may be gradually adjusted to the second position corresponding to the desired section image according to the prediction path determined by the at least two target positions. As shown in FIG. 10, the image data of the first section (the image in one of the areas 108) may be sequentially updated by the image data of the section 105, 106, 107 passing through the target positions (102, 103, 104) on the spatial search path 101, thereby obtaining multiple image data by the adjustment unit 110, until the desired image data is obtained. As shown in FIG. 10, the orientation of the multiple image data of the first section at the multiple second positions (e.g., section image data 105, 106, 107) in the three-dimensional ultrasound volume data may be orthogonal to the spatial search path 101. It is also possible that the orientation of the multiple image data of the first section at the multiple second positions (e.g., section image data 105, 106, 107) in the three-dimensional ultrasound volume data is tangent to the spatial search path 101, which will not be shown in the figure. The positional relationship between the sections 105, 106, 107 indicated in the area 109 in FIG. 10 and the spatial search path 101 may also be regarded as the adjustment display information corresponding to the prediction path (e.g., the schematic diagram of the positional relationship between the sections 105, 106, 107 and the spatial search path 101). The adjustment display information may be output.

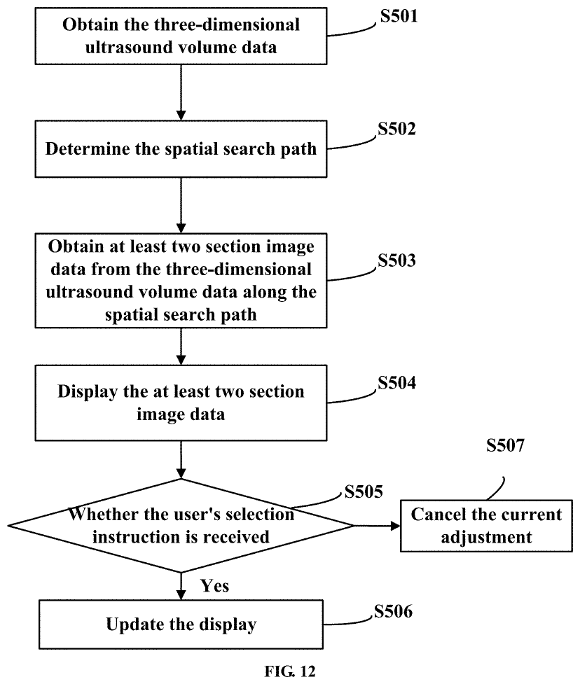

Further, based on the spatial search path above, in one embodiment of the present disclosure, a flexible and simple method for adjusting the position of the section is provided. Referring to FIG. 11 and FIG. 12, the method may include the following steps.

In step S501, a three-dimensional ultrasound volume data may be obtained using the same methods as the embodiments above.

In step S502, a spatial search path may be obtained, which may include at least two target positions. For example, in FIG. 11, three section images, such as those indicated by 118, are displayed on the display interface, with the dashed lines indicating the area of the tissue being examined in the ultrasound image. A three-dimensional ultrasound image may generally be displayed in the area indicated by 119, although a section image may also be displayed therein. A spatial search path 111 (indicated by a black curve with arrow in the figure) is drawn in the area indicated by 119, and multiple target positions (112, 113, 114) may be determined on the spatial search path 111, where the spatial search path 111 passes through the tissue being examined at the multiple target positions (112, 113, 114). The multiple target positions (112, 113, 114) may be determined on the spatial search path by a preset distance (e.g., in an equally spaced manner). Alternatively, specific anatomical structural points (e.g., mitral valve, right ventricular center point, etc.) in the tissue may be identified and the positions on or near the specific anatomical structural points and on the spatial search path may be determined as the target positions. In FIG. 11, the section selected to be adjusted may be displayed as a thick line box (area 118 in the upper left corner in FIG. 11), and the spatial search path 111 drawn in the area 119 may be used to adjust the section corresponding to the area 118 at the upper left corner.

In step S503, at least two section image data may be extracted from the three-dimensional ultrasound volume data along the spatial search path. For example, at least two target positions (112, 113, 114) may be determined on the spatial search path 111, and the image data of the sections 115, 116, 117 at the target position that are tangent or orthogonal to the spatial search path may be obtained as the at least two section image data extracted from the three-dimensional ultrasound volume.

In step S504, the at least two section image data may be displayed to obtain at least two section images 131, 132, 133.

In step S505, it may be determined whether the user's selection instruction is received. If the selection instruction is received, step 506 may be performed, in which an image data may be selected from the at least two section image data to update the displayed image of the section being adjusted (for example, the area 118 in the upper left corner of FIG. 11); otherwise, step 507 may be performed, in which the current adjustment process may be canceled, and the orientation of the section in the three-dimensional ultrasound volume data may be adjusted by changing the spatial search path or by performing the fine adjustment mentioned above by the adjustment unit. The user select an image from the displayed multiple section images 131, 132, 133 to update the image in the area 118 (the area 118 in the upper left corner in FIG. 11). In the present embodiment, the at least two section image data extracted from the three-dimensional ultrasound volume data on the spatial search path 111 may be tangent or orthogonal to the spatial search path in the three-dimensional ultrasound volume data.

As shown in FIG. 11, the image data of the first section at the multiple second positions (e.g., the image data of the sections 115, 116, 117) in the three-dimensional ultrasound volume data may be orthogonal to the spatial search path 111. Alternatively, it may also be possible that the image data of the first section at the multiple second positions (e.g., the image data of the sections 115, 116, 117) in the three-dimensional ultrasound volume data may be tangent to the spatial search path 111, which will not be shown in the figure. The positional relationship between the sections 115, 116, 117 indicated in the area 119 in FIG. 11 and the spatial search path 111 may also be regarded as the adjustment display information corresponding to the prediction path (e.g., the schematic diagram of the positional relationship between the sections 115, 116, 117 and the spatial search path 111). The adjustment display information may be output.

The spatial search path in this embodiment may be obtained according to the input of the user on the image. The image here may be an ultrasound image obtained according to the three-dimensional ultrasound volume data. The ultrasound image may include at least one of a section image and a three-dimensional image. Based on the user's input on the ultrasound image, the spatial search path may be obtained.

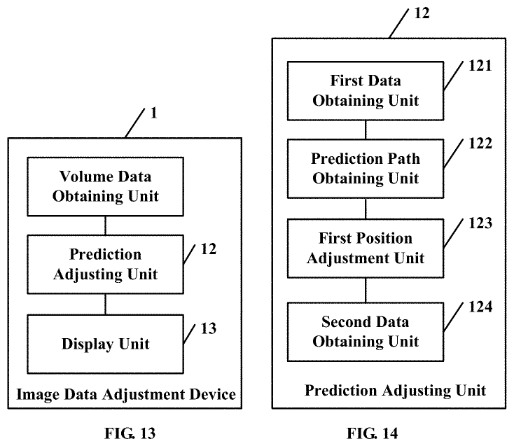

The image data adjustment device in the embodiments of the present disclosure will be described in detail below with reference to FIG. 13 to FIG. 19. It should be noted that the image data adjustment device shown in FIG. 13 to FIG. 19 may be configured to perform the methods of the embodiments shown in FIG. 2 to FIG. 12. For the convenience of description, only the portions related to the present embodiment are shown. Regarding the specific technical details not disclosed, reference may be made to the embodiments shown in FIG. 2 to FIG. 12.

In one embodiment of the present disclosure, an image data adjustment device may include the following units:
a volume data obtaining unit which may be configured to obtain the three-dimensional ultrasound volume data of the examined target body;
a prediction adjustment unit which may be configured to determine a prediction mode of adjusting the orientation of the section in the three-dimensional volume data, and obtain the image data from the three-dimensional ultrasound volume data according to the prediction mode; and
a display unit which may be configured to display the obtained image data.

FIG. 13 is a schematic structural diagram of an image data adjustment device according to one embodiment of the present disclosure. As shown in FIG. 13, the image data adjustment device 1 of the present embodiment may include a volume data obtaining unit 11, a prediction adjusting unit 12, and a display unit 13.

In the present embodiment, as shown in FIG. 14, the prediction adjustment unit 12 may include a first data obtaining unit 121, a prediction path obtaining unit 122, a first position adjustment unit 123, and a second data obtaining unit 124.

The display unit may be configured to display the second image data of the section. Regarding the implementation of the functions of the various units, reference may be made to the detailed description of the various steps in FIG. 2 to FIG. 12, and only a part of the description will be described herein.

The volume data obtaining unit 11 may be configured to obtain the three-dimensional ultrasound volume data of the examined target body.

Specifically, the volume data obtaining unit 11 may obtain the three-dimensional ultrasound volume data of the examined target body. It may be understood that the target body may be a body tissue or organ of a human or an animal, such as a brain tissue or a cardiovascular tissue, etc. The three-dimensional ultrasound volume data may be obtained by scanning the target body using the image data adjustment device 1, such as the intracranial three-dimensional ultrasound volume data of the brain tissue. The three-dimensional ultrasound volume data may also be obtained from another three-dimensional ultrasound imaging system or a server through the network. The three-dimensional ultrasound volume data here may be obtained by scanning using a 2D matrix probe, or by reconstructing a series of two-dimensional ultrasound image data obtained by a 1D linear probe.

The prediction adjustment unit 12 may be configured to determine the prediction mode for adjusting the orientation of the section in the three-dimensional volume data, and obtain the image data from the three-dimensional ultrasound volume data according to the prediction mode. In a specific implementation, the prediction adjustment unit 12 may include:
the first data obtaining unit 121 which may be configured to obtain the first image data of the section at the first position in the three-dimensional ultrasound volume data.

Specifically, the first data obtaining unit 121 may obtain the first image data of the section at the first position from the three-dimensional ultrasound volume data. It can be understood that the first position may be a display position of the first image data in the three-dimensional ultrasound volume data when the image data adjustment device 1 obtain the three-dimensional ultrasound volume data by scanning. The first image data of the section may be an image data representing a specific cross section of the body tissue related to a human or animal body anatomical orientation in the three-dimensional ultrasound volume data. For example, the first image data of the section may be the image data of the cerebellar section in an intracranial three-dimensional ultrasound volume data obtained by scanning a brain tissue. The first image data of the section may include the image data of at least one section. In some embodiments of the present disclosure, the section may be a section corresponding to any orientation in the three-dimensional ultrasound volume data. For example, taking the image of the brain or cardiac tissue as an example, the section may be any one of a cerebellar section, a thalamic section, a lateral ventricle section, a median sagittal section, a four-chamber heart section, a left ventricular outflow tract section, a right ventricular outflow tract section, a three-vessel tracheal section, a gastric vesicle section and an arterial catheter arch section, etc., and a combination thereof.

In some embodiments of the present disclosure, the first data obtaining unit 121 may automatically obtain the first image data of the section at the first position from the three-dimensional ultrasound volume data. The automatic obtaining may be implemented with an automatic calculation of the calculation program, in which a certain section image data may be obtained using an automatic segmentation algorithm of the image. For example, the first image data of the median sagittal section may be automatically obtained from the ultrasound image according to the image characteristics based on the spatial orientation of the brain and the characteristics of the brain tissue.

The prediction path obtaining unit 122 may be configured to obtain the prediction path when the adjustment instruction output by the adjustment unit is obtained.

It should be noted that, based on long-term clinical experience, each specific section image in a three-dimensional ultrasound volume data of the body tissue may correspond to an most or more frequently used adjustment mode, that is, to a most or more likely adjustment path (which is referred to as prediction path herein). The specific section may be a diagnostic section commonly used by doctors, or the section indicated in a standard medical examination procedure. The prediction path may be a transformation including one of a translation in the X, Y, and Z direction and a rotation in the X, Y, and Z directions or a combination thereof. For example, regarding the four-chamber heart section, the three-vessel tracheal section or the gastric vesicle section, etc. which are transverse section, the prediction path may be a translation in the Z direction; regarding the left ventricular outflow tract section, the prediction path may be a rotation in the Y direction; regarding the right ventricular outflow tract section and the arterial catheter arch section, the prediction path may be a rotation in the Z direction; regarding the median sagittal section, the prediction path may be a translation in the Y direction; and so on. Besides indicating which operation will be performed in which direction, the prediction path may also include the step size of the operation performed in the direction. For example, regarding the left ventricular outflow tract section, the prediction path may be a rotation in the Y direction by 1 degree; regarding the right ventricular outflow tract section and the arterial catheter arch section, the prediction path may be a rotation in the Z direction by 2 degrees; regarding the median sagittal section, the prediction path may be a translation in the Y direction by 2 units; and so on. It can be understood that the prediction path may include a combination of at least one of the moving direction and the operation mode and a moving range (the moving range may include a distance and/or an angle). The prediction path corresponding to each specific section may be stored in the image data adjustment device, that is, the prediction path corresponding to each section image data may be known. In the embodiments of the present disclosure, it will not be limited to the specific section, but may be applied to the adjustment for any section. That is to say, the prediction path corresponding to each section orientation may be stored in the image data adjustment device.

It can be understood that, when the orientation of the section in the three-dimensional ultrasound volume data is different, the prediction path corresponding to the section will be different. The section image desired to be observed in the three-dimensional ultrasound volume data may not be determined in one time. For example, the first position corresponding to the four-chamber heart section automatically obtained from the three-dimensional ultrasound volume data of the heart may be offset to the left or right relative to the desired position thereof. If the four-chamber heart section is to be adjusted to a desired position suitable for observation (for example, a central position in the three-dimensional ultrasound volume data of the heart chamber), an auxiliary manual operation may be desired to participate in the adjustment. When the four-chamber heart section is offset to the left, the corresponding prediction path may be a translation to the right. When the four-chamber heart section is offset to the right, the corresponding prediction path may be a translation to the left. Usually, six knobs or buttons are used in the ultrasound system to perform manual adjustment of the section. The six knobs or buttons are X-axis translation, X-axis rotation, Y-axis translation, Y-axis rotation, Z-axis translation and Z-axis rotation. Therefore, it will be desired that the user has a very clear understanding to the difference between the image space and the physical space, and uses a combination operation of the six knobs or buttons to obtain the desired section image. This is very complicated and requires a good understanding to medical anatomy. Moreover, it is also necessary to be very familiar with the correspondence between the spatial orientation of the sections and the anatomical structures. Therefore, the difficulty and complexity of the use of the ultrasound device are increased. Based on this issue, in the present embodiment, the automatic obtainment or configuration of the prediction path may be activated according to the adjustment instruction inputted by the user through the adjustment unit to obtain the prediction path corresponding to the section, thereby reducing the number of buttons, decreasing the complexity of the operation, increasing the intelligence of the machine, reducing the cost of hardware, and achieving more miniaturization.

Further, when the orientation of the section in the three-dimensional ultrasound volume data is different, even the prediction path obtained according to the adjustment instruction inputted by the same adjustment unit will be different, because the prediction path corresponding to the adjustment unit will be automatically configured for different section. For example, in the case that the section is a four-chamber heart section, the prediction path based on the input inputted by a virtual button on the interface will be a translation in the Z-direction, while in the case that the section is the left ventricular outflow tract section, the prediction path based on the same input inputted by the virtual button will be a rotation in the Y-direction.

It can be understood that the adjustment instruction may be a control instruction which is inputted by the medical personnel through the adjustment unit of the image data adjustment device 1 and triggers the three-dimensional ultrasound volume data. The adjustment unit may be a virtual adjustment unit or a physical adjustment unit. The virtual adjustment unit may include any one of a key, a button and a slide bar arranged on the display interface of the section image data. The physical adjustment unit may be a hardware having a substantial shape, such as any one of physical hardware buttons, keys, knobs, scroll wheels and mouse.

Specifically, when obtaining the adjustment instruction inputted through the human-computer interaction device (that is, the adjustment unit), the prediction path obtaining unit 122 may obtain the prediction path. For example, when the image data adjustment device 1 performs a three-dimensional ultrasound examination on a human heart, the prediction path obtaining unit 122 may obtain a prediction path of a section (four-chamber heart section) in the Z direction in the obtained three-dimensional ultrasound volume data.

The first position adjustment unit 123 may be configured to adjust the section from the first position to the second position in the three-dimensional ultrasound volume data along the prediction path.

Specifically, the first position adjustment unit 123 may adjust the section from the first position to the second position in the three-dimensional ultrasound volume data along the prediction path. It can be understood that the second position may be a final display position of the section in the three-dimensional ultrasound volume data after the section is adjusted from the first position in the three-dimensional ultrasound volume data according to the prediction path. It can be understood that the first position adjustment unit 123 may adjust the four-chamber heart section from the first position to the second position in the three-dimensional ultrasound volume data of the heart according to the translation in the Z-direction corresponding to the image data of the section (four-chamber heart section). In one embodiment, the prediction path may include any one of a prediction path of moving a preset distance in one direction and a prediction path of moving preset distances in at least two directions. When inputting the adjustment instruction through the adjustment unit, each time the adjustment instruction is input, the corresponding prediction path may be moving a preset distance in one direction or moving preset distances in at least two directions. The preset distance here may be measured in angle and/or displacement.

The second data obtaining unit 124 may be configured to obtain the second image data of the section at the second position in the three-dimensional ultrasound volume data.

It can be understood that in the process of adjusting the section at the first position in the three-dimensional ultrasound volume data according to the prediction path corresponding to the first image data, the image data adjustment device 1 may display in real time the image data of the section in the three-dimensional ultrasound volume data during the adjustment on the display screen. Optionally, the image data adjustment device 1 may not display the adjustment process of the section, but directly display the section at the final position reached when the adjustment is completed, that is, at the second position. When the adjustment is completed and the section reaches the final position (that is, the second position), the image data adjustment device 1 may display the image data of the section at the second position in the three-dimensional ultrasound volume data on the display screen, that is, display the second image data of the section.

Specifically, when the adjustment is completed, the second data obtaining unit 124 may obtain the second image data of the section at the second position in the three-dimensional ultrasound volume data. It can be understood that the second image data may be an image data in the cross section at the second position corresponding to the section at the first position. For example, the first image data at the first position is an image data of the four-chamber heart section, and the second image data at the second position is an image data of the four-chamber heart section translated in the Z-direction.

The display unit 13 may be configured to display the second image data to obtain the section image.

Specifically, the image data adjustment device 1 may display the content of the second image data on the current display screen, such as displaying the image data of the four-chamber heart section translated in the Z direction. The image data of the section displayed in the display unit 13 may not necessarily be the final desired image data of the section, but may be an image data during the process of obtaining the desired image data of the section. That is, in the embodiment of the present disclosure, the second position corresponding to the desired image data of the section may be obtained by one input through the adjustment unit, or be obtained through adjustments to multiple second positions based on multiple inputs through the adjustment unit. Therefore, the prediction path in this embodiment will not be limited to adjust the section to the second position corresponding to the desired image data by one adjustment. Rather, in the prediction path in this embodiment, it may also be possible that the section is adjusted from the first position to the second position corresponding to the desired image data through step adjustments for gradually approaching the second position. The step adjustments may be performed according to the prediction direction and/or operation obtained by prior knowledge, thereby saving the adjustment time and reducing the adjustment complexity.

In the embodiments of the present disclosure, the three-dimensional ultrasound volume data of the detection target body may be obtained, and the first image data of the section at the first position may be obtained in the three-dimensional ultrasound volume data. When the adjustment instruction outputted by the adjustment unit is obtained, the prediction path corresponding to the first image data may be obtained, and the section may be adjusted from the first position to the second position in the three-dimensional ultrasound volume data along the prediction path. Thereafter, the second image data of the section at the second position in the three-dimensional ultrasound volume data may be obtained and displayed. By automatically obtaining the prediction path corresponding to the first image data and automatically adjusting the section at the first position in the three-dimensional ultrasound volume data according to the prediction path to obtain the second section image data, the complexity of obtaining a specific section image in the three-dimensional ultrasound volume data is reduced.

FIG. 15 is a schematic structural diagram of another image data adjustment device in one embodiment of the present disclosure. As shown in FIG. 15, the image data adjustment device 1 may include a volume data obtaining unit 11, a prediction adjusting unit 12, a display unit 13, a section type obtaining unit 14, a path set unit 15, and a preset storage unit 16.

The volume data obtaining unit 11 may be configured to obtain the three-dimensional ultrasound volume data of the examined target body.

Specifically, the volume data obtaining unit 11 may obtain the three-dimensional ultrasound volume data of the examined target body. It may be understood that the target body may be a tissue or organ of a human or an animal, such as brain tissue or cardiovascular tissue, etc. The three-dimensional ultrasound volume data above may be a three-dimensional ultrasound volume data obtained by scanning the target body using the image data adjustment device, such as an intracranial three-dimensional ultrasound volume data obtained by scanning the brain tissue.

The prediction adjustment unit 12 may be configured to determine the prediction mode for adjusting the orientation of the section in the three-dimensional volume data, and obtain the image data from the three-dimensional ultrasound volume data according to the prediction mode. In a specific implementation, the prediction adjustment unit 12 may include a first data obtaining unit 121, a prediction path obtaining unit 122, a first position adjustment unit 123, and a second data obtaining unit 124. Regarding the specific implementation, reference may be made to the methods or devices above, which will not be described here again.

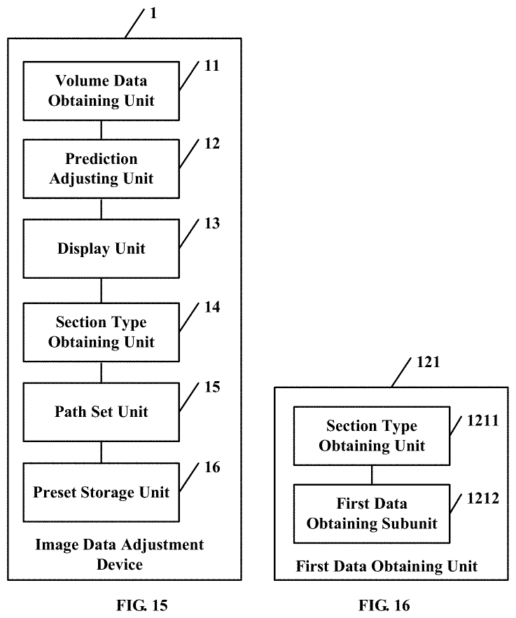

FIG. 16 is a schematic structural diagram of the first data obtaining unit in one embodiment of the present disclosure. As shown in FIG. 16, the first data obtaining unit 121 may include:
a section type obtaining subunit 1211 which may be configured to obtain an inputted section type.

Specifically, the section type obtaining subunit 1211 may obtain the inputted section type. It can be understood that the section type may be a type name or a type number that represents the type of the section image data. For example, a type name of “four-chamber heart section” or a type number of “01” which is pre-agreed to represent the four-chamber heart section inputted by voice may be obtained. IN another embodiment, the section type obtaining subunit 1211 may obtain the orientation of the section inputted by the user. The section type may be a specific form of the orientation of the section.
a first data obtaining subunit 1212 which may be configured to automatically obtain the first image data of the section located at the first position from the three-dimensional ultrasound volume data;

Specifically, the first data obtaining subunit 1212 may automatically obtain the first image data at the first position from the three-dimensional ultrasound volume data according to the section type. It can be understood that the first position may be a display position of the first image data in the three-dimensional ultrasound volume data when the image data adjustment device obtain the three-dimensional ultrasound volume data by scanning. The first image data may be an image data representing a cross section of the body tissue related to a human or animal body anatomical orientation in the three-dimensional ultrasound volume data. For example, the first image data of the section may be the image data of the cerebellar section in an intracranial three-dimensional ultrasound volume data obtained by scanning a brain tissue. Alternatively, the first data obtaining subunit 1212 may automatically obtain the first image data of the section at the first position from the three-dimensional ultrasound volume data according to the orientation of the section.

The section type obtaining unit 14 may be configured to obtain the section type of at least one specific section corresponding to the first image data;

It can be understood that the first image data may include the image data of at least one cross section. For example, the first image data in the intracranial three-dimensional ultrasound volume data may include the image data of the cerebellar section, the thalamic section or the lateral ventricle section, etc. It should be noted that the sections of different types may have different orientations in the three-dimensional volume data. For example, the cerebellar section may be in an upper orientation in the intracranial three-dimensional ultrasound volume data, while the thalamic section may be in a lower orientation in the intracranial three-dimensional ultrasound volume data. It may also be possible that the section type obtaining unit 14 is configured to obtain at least one orientation corresponding to the first image data.

Specifically, the section type obtaining unit 14 may determine the section type of the cross sections in the at least one cross section corresponding to the first image data according to the image data of each cross section. For example, in the cast that the image data of the cross section indicates that it is an image of the cerebellum, it may be determined that section type of the cross section is cerebellar section.

The path set unit 15 may be configured to configure at least one prediction path corresponding to the at least one cross section according to the section type of the at least one cross section.

Specifically, the path set unit 15 may set at least one prediction path corresponding to the at least one cross section according to the section type of the at least one cross section. It can be understood that the path set unit 15 may set the prediction paths corresponding to the cross sections according to the section type of the cross sections of the at least one cross section. For example, in the case that the section type of a certain cross section in the at least one cross section is four-chamber heart section, the path set unit 15 may set the prediction path of Z-direction translation for such cross section according to long-term clinical experience. It will be appreciated that each cross section in the at least one cross section may correspond to a prediction path obtained based on long-term clinical experience that is most frequently used. In one embodiment, the path set unit 15 may set the at least one prediction path corresponding to the at least one cross section according to the orientation of the at least one cross section.

The preset storage unit 16 may be configured to pre-store the orientations of the multiple section image data in the three-dimensional ultrasound volume data and the prediction paths corresponding to the orientations.

Specifically, the preset storage unit 16 may pre-store the orientations of the multiple cross sections in the three-dimensional ultrasound volume data and the prediction paths corresponding to the orientations. For example, the preset storage unit 16 may pre-store the upper middle orientation of the median sagittal section in the three-dimensional volume data and the prediction path of translation in the negative Y-direction corresponding to such orientation.

It can be understood that the different orientations of the cross section image data in the three-dimensional ultrasound volume data may correspond to different section types. The prediction path may be directly searched according to the value representing the orientation, or according to the section type. For different orientations of the first image data in the three-dimensional ultrasound volume data, the prediction paths corresponding to the first image data will be different. For example, due to the difference in acquiring the three-dimensional ultrasound volume data, the orientation of the obtained first image data in the three-dimensional ultrasound volume data is different (for example, the position of the four-chamber heart section in the three-dimensional ultrasound volume data of the heart may be on the left or right). When it is desired to adjust the four-chamber heart section to a position suitable for observation (for example, the middle position in the three-dimensional ultrasound data of the heart), the prediction path will be translation to the right when the four-chamber heart section is on the left and be translation to the left when the four-chamber heart section is on the right.

Further, for different orientations of the first image data in the three-dimensional ultrasound volume data, even the prediction paths obtained according to the adjustment instruction inputted by the same adjustment unit may be different. For example, when the first image data is a four-chamber section image data, the prediction path obtained according to the input of the virtual button on the interface may be a translation in the Z-direction, while, when the first image data is a left ventricular outflow tract section image data, the prediction path obtained according to the input of the same virtual button may be a rotation in the Y-direction.

In the embodiment of the present disclosure, by storing the multiple section image data and corresponding prediction paths in advance, the accuracy of automatically obtaining the prediction path according to the section image can be increased.

In a possible implementation of the embodiment of the present disclosure, the prediction path obtaining unit 122 may be configured to obtain at least one prediction path corresponding to the at least one cross section when the adjustment instruction input by the at least one adjusting unit is obtained. Specifically, in one embodiment, the prediction path obtaining unit 122 may search for the prediction path corresponding to the first image data of the section. For example, the prediction path may be searched according to the orientation of the first image data of the section in the three-dimensional ultrasound volume data. Alternatively, the adjustment instruction outputted by the adjustment unit and the searched prediction path may be associated, and the adjustment unit may be reconfigured by the searched prediction path. Each time the orientation of the first image data of the section is changed, the prediction path may be associated by reconfiguring the adjustment unit. Therefore, the complexity of adjusting the section may be optimized, and the section may be conveniently and quickly adjusted to the desired position.

It can be understood that the number of the adjustment units in the image data adjustment device 1 may be the same as the number of the cross sections currently displayed. That is, in the case that the image data adjustment device has four adjustment units, the display screen of the image data adjustment device 1 may be divided into four regions to display the image data of four cross sections. It can be understood that the prediction path obtaining unit 122 may obtain at least one prediction path corresponding to the at least one cross sections according to the adjustment instruction inputted by at least one adjustment unit, and may accordingly perform adjustment on the three-dimensional ultrasound volume data according to the prediction path corresponding to each cross section.

Specifically, when obtaining the adjustment instruction inputted by the user through the at least one adjustment unit, the prediction path obtaining unit 122 may obtain at least one prediction path corresponding to the at least one cross section. As shown in FIG. 4, there are 4 cross sections (the four-chamber heart section, the arterial catheter arch section, the left ventricular outflow tract section and the right ventricular outflow tract section) on the display, and the prediction paths corresponding to the cross sections are Z-direction translation, Z-direction rotation, Y-direction rotation and Z-direction rotation, respectively.

In the implementation of the embodiments of the present disclosure, the image data adjustment device 1 may have one or more adjustment units.

Optionally, in the case that the image data adjustment device has one adjustment unit (as shown in FIG. 5a, the virtual button A on the display screen of the image data adjustment device 1), the one adjustment unit may be configured to perform the adjustment in any direction, manner and distance. For example, with the virtual button A, a movement of preset distance in one direction may be achieved, in which the movement of preset distance may include a translation of preset distance and a rotation of preset angle (e.g., a translation of 1 mm in X direction or a rotation of 1 degree around X axis), or a movement of preset distance in at least two directions may be achieved (e.g., a translation of 1 mm in X direction and a translation of 1 mm in Y direction).

Optionally, in the case that the image data adjustment device has two adjustment units (as shown in FIG. 5b, the virtual sliding bar B and the virtual button C on the display screen of the image data adjustment device 1), the two adjustment units may correspond to two adjusting manners. For example, the virtual sliding bar B may be configured to perform the translation adjustment in the X, Y and Z directions, and the virtual button C may be configured to perform the rotation adjustment around the X, Y and Z directions.

Optionally, in the case that the image data adjustment device has three adjustment unit (as shown in FIG. 5c, the virtual button D, the virtual knob E and the virtual sliding bar F on the display screen of the image data adjustment device 1), the three adjustment units may respectively correspond to the adjustment in three directions. For example, adjusting the virtual button D may achieve a movement of preset distance in the X direction, adjusting the virtual knob E may achieve a movement of preset distance in the Y direction, and adjusting the virtual sliding bar F may achieve a movement of preset distance in the Z direction.

The first position adjusting unit 123 may be configured to adjust the section from the first position to the section position in the three-dimensional ultrasound volume data along the at least one prediction path.

Specifically, the first position adjusting unit 123 may adjust the cross section from the first position to the second position in the three-dimensional ultrasound volume data along the at least one prediction path. As shown in FIG. 4, the image data adjustment device may adjust the cross section from the first position to the second position in the three-dimensional ultrasound volume data simultaneously according to the Z-direction translation corresponding to the four-chamber heart section, the Z-direction rotation corresponding to the arterial catheter arch section, the Y-direction rotation corresponding to the left ventricular outflow tract section and the Z-direction rotation corresponding to the right ventricular outflow tract section.

In the embodiments of the present disclosure, the prediction path may usually be one of the six basic adjustment modes of rotation and translation in the X, Y and Z directions. That is, the dimensionality reduction method from 6-dimensional space to 1-dimensional space may be used, in which one certain dimension of the six dimensions is directly taken according to the orientation of the cross section in the human anatomy. In other embodiments, the dimensionality reduction method may also be a linear or non-linear combination of the 6-dimensional parameters, such as a combination of translations in X and Y direction in which the translations in X and Y direction may be achieved simultaneously when the corresponding adjustment unit is adjusted. The dimensionality reduction may also be implemented according to the anatomical features of the cross section using a machine learning method. For example, the user's usual operating habits may be recorded by the machine and stored as data, and the most common operation path of the user may be obtained therefrom by the machine learning algorithm. Such most common operation path may be the most likely prediction path. The commonly used machine learning algorithms may be support vector machine (SVM), principal component analysis (PCA), convolutional neural network (CNN), recurrent neural network (RNN) and the like.

It can be understood that the image data adjustment device 1 may adjust the cross section at the first position in the three-dimensional ultrasound volume data using the prediction path obtained according to any one of the six dimensional spatial parameters, the linear or non-linear combination of the six dimensional spatial parameters or the conventional adjustment path obtained by the machine learning, etc.

The second data obtaining unit 124 may be configured to obtain the second image data of the section located at the second position in the three-dimensional ultrasound volume data.

Optionally, during the movement of the cross section in the first position in the three-dimensional ultrasound volume data according to the prediction path corresponding to the first image data, the change of the first image data in the three-dimensional ultrasound volume data may be displayed on the display screen of the image data adjustment device 1 in real time. Optionally, the display screen of the image data adjustment device 1 may also not display the adjustment process of the cross section at the first position, but directly display the image data at the final position reached when the adjustment is completed, that is, at the second position. When the adjustment is completed and the cross section reaches the final position, that is, the second position, the second data obtaining unit 124 may display the state of the cross section at the second position in the three-dimensional ultrasound volume data, that is, display the second image data of the cross section.

Specifically, when the adjustment is completed, the second data obtaining unit 124 may obtain the second image data of the cross section at the second position in the three-dimensional ultrasound volume data. It can be understood that the second image data may be an image data of the cross section at the second position corresponding to the first image data. For example, the first image data at the first position is the image data of the four-chamber heart section, and the second image data at the second position is the image data of the four-chamber heart section translated in the Z direction. Further, in the case that the first image data corresponds to at least one cross section, the second image data may also correspond to at least one cross section.

The display unit 13 may be configured to display the second image data to obtain the section image.

Specifically, the image data adjustment device 1 may display the image data content indicated by the second image data of the section in the current display screen, such as displaying the image data of the four-chamber heart section translated in the Z-direction. The section image displayed on the display unit 13 may not be necessarily the final desired image data of the section, but may be an image obtained during the process of obtaining the desired image data of the section. That is, in the embodiments of the present disclosure, the section may be directly adjusted to the second position corresponding to the desired image data of the section by one input of the adjustment unit. Alternatively, the desired image data of the section may be obtained by multiple inputs of the adjustment unit through multiple second positions. Therefore, in the present embodiment, the prediction path will not be limited to adjust the section to the second position corresponding to the desired image data by one time. Alternatively, in the present embodiment, with the prediction path, the section may also be adjusted from the first position to the second position corresponding to the desired image data of the section by a step adjustment by which the section gradually reaches to the second position. The step adjustment may be performed according to the prediction direction and/or operation obtained by prior knowledge, thereby saving the adjustment time and reducing the adjustment complexity.

In the embodiments of the present disclosure, the section at the first position in the three-dimensional ultrasound volume data may be adjusted according to at least one prediction path corresponding to the at least one cross section corresponding to the first image data. Therefore, the diversity of the adjustment to the cross section in the three-dimensional ultrasound volume data may be increased.

In the embodiments of the present disclosure, the three-dimensional ultrasound volume data of the examined target body may be obtained, and the first image data of the section at the first position may be obtained from the three-dimensional ultrasound volume data. When the adjustment instruction output by the adjustment unit is acquired, the prediction path may be obtained, and the section at the first position may be adjusted to the second position in the three-dimensional ultrasound volume data along the prediction path. Thereafter, the second image data of the section at the second position in the three-dimensional ultrasound volume data may be obtained, and displayed. By automatically obtaining the prediction path corresponding to the first image data and automatically adjusting the section at the first position in the three-dimensional ultrasound volume data according to the prediction path to obtain the second image data of the section, the complexity of adjusting the cross section in the three-dimensional ultrasound volume data may be reduced. By pre-storing the multiple section image data and their corresponding prediction paths, the accuracy of automatically obtaining the prediction path according to the section image may be increased. By adjusting the section at the first position in the three-dimensional ultrasound volume data according to at least one prediction path corresponding to the at least one cross section corresponding to the first image data, the diversity of the adjustment to the cross section in the three-dimensional ultrasound volume data may be increased.

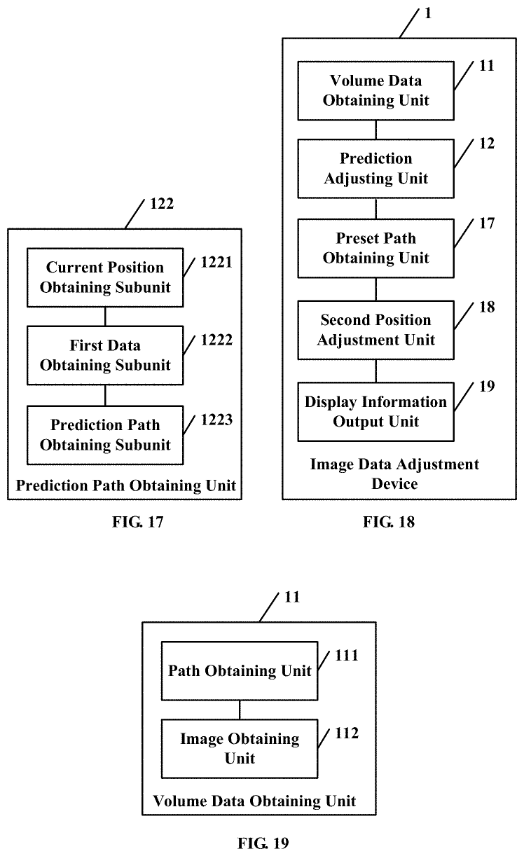

In a possible implementation of the embodiment of the present disclosure, the prediction path obtaining unit 122 may include the following units, as shown in FIG. 17:
a current position obtaining sub-unit 1221 which may be configured to obtain a current position where the indication identifier is located in the current screen;

It can be understood that multiple section image data may be simultaneously displayed on the display screen of the image data adjustment device 1. When the multiple image data are simultaneously displayed on the display screen, the image data adjustment device 1 may perform the adjustment on one of the multiple section image data.

Specifically, the current position obtaining sub-unit 1221 may obtain the current position of the indication identifier in the current screen. It may be understood that the indication identifier may be the cursor in the current screen, and the user may place the cursor on the position of the section image data to be adjusted of the multiple section image data displayed on the current screen to activate the section image data to be adjusted. Thereby, the processor can obtain the current position of the cursor. It can be understood that the current position of the indication identifier may be the position of the selected first image data.
a first data obtaining sub-unit 1222 which may be configured to obtain the first image data of the section at the current position;

Specifically, the first data obtaining sub-unit 1222 may obtain the first image data of the section at the current position. As shown in FIG. 7a, when the current position of the cursor is the position of a first section, such as the four-chamber heart section, the first data obtaining sub-unit 1222 may obtain the first image data at such position, that is, the image data of the four-chamber heart section. Optionally, the image data adjustment device 1 may display only the currently selected first image data of the section through the display screen after selecting the first section image data, as shown in FIG. 7b.
a prediction path obtaining sub-unit 1223 which may be configured to obtain the prediction path corresponding to the first section image data at the current position when the adjustment instruction inputted through the adjustment unit is obtained;

Specifically, when the human-machine interaction module (i.e. the adjustment unit) in the image data adjustment device 1 obtains the adjustment instruction input by the user through the adjustment unit, the prediction path obtaining sub-unit 1223 may obtain the prediction path corresponding to the first image data at the current position. It can be understood that the position of the first image data in the three-dimensional ultrasound volume data and the corresponding prediction path have been stored in the image data adjustment device, and when the user activates the adjustment unit to adjust the section, the processor may directly retrieve the corresponding prediction path from the memory.

In the embodiments of the present disclosure, the first image data of the section may be selected by the cursor in the current screen, and the prediction path corresponding to the first image data may be obtained, thereby avoiding the adjustment to the section which needs not to be adjusted. Therefore, the unnecessary adjustment may be reduced, and the adjustment efficiency may be increased.

FIG. 18 is a schematic structural diagram of another image data adjustment device in one embodiment of the present disclosure. As shown in FIG. 18, the image data adjustment device 1 of the present embodiment may include a volume data obtaining unit 11, a prediction adjustment unit 12, a preset path obtaining unit 17, a second position adjustment unit 18, and a display information output unit 19.

Regarding the specific implementation of the volume data obtaining unit 11 and the prediction adjustment unit 12, reference may be made to the related description of the methods or devices in the embodiments above, which will not be described again.

The preset path obtaining unit 17 may be configured to obtain a preset prediction path that is input according to a preset manner.

It can be understood that, in the embodiments of the present disclosure, the image data adjustment device 1 may determine the prediction path by the interaction with the user. For example, the user may draw a spatial search curve corresponding to a specific cross section of the fetal heart as shown in FIG. 9 in a certain manner. When the adjustment unit is activated, the orientation of the corresponding section may be adjusted along such curve, where the searched section may be orthogonal or tangent to the user-defined curve.

Specifically, the preset path obtaining unit 17 may obtain the preset prediction path that is input by the user according to a preset manner. It can be understood that the preset manner may be a definition of a spatial search curve implemented by an algorithm or manually drawing a spatial search curve by a cursor, such as the spatial search curve manually drawn by the cursor as shown in FIG. 9. The preset prediction path may be the custom spatial search curve.

The second position adjustment unit 18 may be configured to adjust the section from the first position to the second position in the three-dimensional ultrasound volume data along the preset prediction path.

Specifically, the second position adjustment unit 18 may adjust the section from the first position to the second position in the three-dimensional ultrasound volume data along the prediction path. For example, the section at the first position in the three-dimensional ultrasound volume data may be adjusted according to the spatial search curve corresponding to the specific cross section of the fetal heart as shown in FIG. 9.

In the embodiments of the present disclosure, the customized prediction path may be obtained, and the section at the first position in the three-dimensional ultrasound volume data may be adjusted according to the customized prediction path. Therefore, the accuracy of the adjustment can be increased.

The display information output unit 19 may be configured to generate the adjustment display information corresponding to the prediction path and output the adjustment display information.

It can be understood that, because the prediction paths of different cross sections are different, the display information output unit 19 may generate the adjustment display information corresponding to the prediction paths so as to facilitate the understanding of the user. It can be understood that the adjustment display information may be a text, an icon, or other prompt information capable of informing the user the specific motion direction corresponding to the current prediction path. The adjustment display information may be the prompt information shown in FIG. 4, FIG. 7a and FIG. 7b.

Furthermore, the display information output unit 19 may output the adjustment display information, such as display the prompt information shown in FIG. 4, FIG. 7a and FIG. 7b simultaneously with the second image data on the current display screen.

In the embodiments of the present disclosure, the specific movement directions during the adjustment may be displayed by the adjustment display information. Therefore, the degree of visualization of the adjustment process may be increased.

In the embodiments of the present disclosure, the three-dimensional ultrasound volume data of the examined target body may be obtained, and the first image data of the section at the first position may be extracted from the three-dimensional ultrasound volume data. Based on the preset prediction path inputted according to the preset manner, the section may be adjusted from the first position to the second position in the three-dimensional ultrasound volume data along the prediction path, and the second image data of the section at the second position in the three-dimensional ultrasound volume data may be obtained and displayed. In addition, the adjustment display information corresponding to the prediction path may be generated, and output. By obtaining the customized prediction path and adjusting the section at the first position in the three-dimensional ultrasound volume data according to the customized prediction path, the accuracy of the adjustment is increased. By presenting the movement directions during the adjustment through the adjustment display information, the degree of visualization of the adjustment process is increased.

In another embodiment of the present disclosure, the image data adjustment device 1 may include the volume data obtaining unit 11. As shown in FIG. 19, the volume data obtaining unit 11 may include:
a path obtaining unit 111 which may be configured to obtain the spatial search path, where the spatial search path may include at least two target positions;
an image obtaining unit 112 which may be configured to obtain at least two section image data from the three-dimensional ultrasound volume data along the spatial search path.

The display unit may be configured to display the at least two section image data.

Regarding the specific implementation of the functions of the units above, reference may be made to the detailed description of the steps in the process shown in FIG. 12, which will not be described again here.

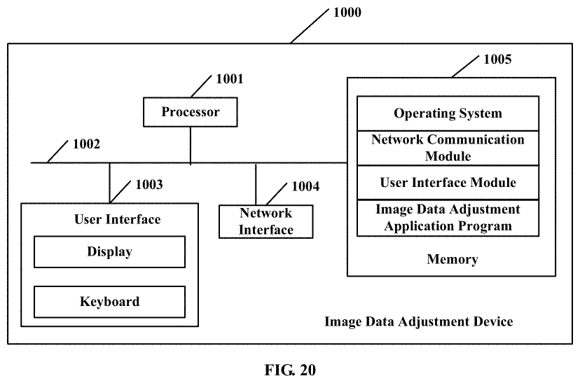

FIG. 20 is a schematic block diagram of an image data adjustment device on another embodiment of the present disclosure. As shown in FIG. 20, the image data adjustment device 1000 may include at least one processor 1001, such as a CPU, at least one network interface 1004, a user interface 1003, a memory 1005, and at least one communication bus 1002. The communication bus 1002 may be configured to implement the communication between these components. The user interface 1003 may include a display and a keyboard. Optionally, the user interface 1003 may also include a standard wired or wireless interface. The network interface 1004 may include a standard wired or wireless interface (such as a WI-FI interface). The memory 1005 may be a high-speed RAM memory, or may be a non-volatile memory, such as at least one disk memory. The memory 1005 may also be at least one storage device located away from the processor 1001. As shown in FIG. 20, the memory 1005 as a computer storage medium may include an operating system, a network communication module, a user interface module, and an image data adjustment application program.

In the image data adjustment device 1000 shown in FIG. 20, the user interface 1003 may be mainly configured to provide an input interface for the user to obtain the data input by the user. The network interface 1004 may be configured to perform data communication with the user terminal. The processor 1001 may be configured to execute the image data adjustment application program stored in the memory 1005 to performing the steps of:
determining a prediction mode for adjusting an orientation of a section in the three-dimensional ultrasound volume data;
obtaining an image data from the three-dimensional ultrasound volume data according to the prediction mode; and
displaying the obtained image data.

In one embodiment, the processor may determine the prediction mode for adjusting the orientation of the section in the three-dimensional ultrasound volume data, obtain the image data from the three-dimensional ultrasound volume data according to the prediction mode and display the obtained image data by:
obtaining a first image data of the section at a first position in the three-dimensional ultrasound volume data;
obtaining a prediction path corresponding to the first image data when an adjustment instruction inputted by the adjustment unit is obtained;
adjusting the section from the first position to a second position in the three-dimensional ultrasound volume data along the prediction path;
obtaining a second image data of the section at the second position in the three-dimensional ultrasound volume data; and
displaying the second image data on the display.

In one embodiment, the prediction paths corresponding to different orientations of the first image data in the three-dimensional ultrasound volume data may be different.

In one embodiment, the prediction paths obtained according to the adjustment instruction inputted by the same adjustment unit for different orientations of the first image data in the three-dimensional ultrasound volume data may be different.

In one embodiment, the prediction path may include any one of an adjustment path of moving a preset distance in one direction and an adjustment path of moving a preset distance in at least two directions.

In one embodiment, the processor 1001 may obtain the first image data of the section at the first position in the three-dimensional ultrasound volume data by:
obtaining a section type inputted;
automatically obtaining the first image data of the section at the first position from the three-dimensional ultrasound volume data according to the inputted section type.

In one embodiment, the first image data may include at least one cross section, and after obtaining the first image data of the section at the first position in the three-dimensional ultrasound volume data, the processor 1001 may be configured to:
obtain a section type of the at least one cross section corresponding to the first image data;
configure at least one prediction path corresponding to the at least one cross section according to the section type of the at least one cross section.

In one embodiment, the processor 1001 may be configured to:
pre-store the orientations of multiple cross sections in the three-dimensional ultrasound volume data and the prediction paths corresponding to the orientations.

In one embodiment, the section type may be used to represent the orientation.

In one embodiment, the processor 1001 may obtain the prediction path when the adjustment instruction output by the adjustment unit is obtained by:
obtaining at least one prediction path corresponding to the at least one cross section when the adjustment instruction inputted through at least one adjustment unit is obtained.

In one embodiment, the processor 1001 may adjust the section from the first position to the second position in the three-dimensional ultrasound volume data along the prediction path by:
adjusting the section from the first position to the second position in the three-dimensional ultrasound volume data along the at least one prediction path.

In one embodiment, the processor 1001 may obtain the prediction path when the adjustment instruction output by the adjustment unit is obtained by:
obtaining a current position of an indication identifier on a current screen;
obtaining the first image data at the current position; and
obtaining the prediction path corresponding to the first image data at the current position when the adjustment instruction output by the adjustment unit is obtained.

In one embodiment, the processor 1001 may be configured to:
obtain the prediction path inputted in a preset manner;
adjusting the section from the first position to the second position in the three-dimensional ultrasound volume data along the prediction path.

In one embodiment, the processor 1001 may, after displaying the second image data, be further configured to:
generate an adjustment display information corresponding to the prediction path, and output the adjustment display information.

In one embodiment, the adjustment unit may be a virtual adjustment unit and/or a physical adjustment unit. The virtual adjustment unit may include any one of a key, a button and a slide bar arranged on the display interface of the section image data, and the physical adjustment unit may include any one of physical hardware buttons and keys.

In one embodiment, the processor 1001 may obtain the first image data of the section at the first position in the three-dimensional ultrasound volume data by:
obtaining an orientation of the section inputted; and
automatically obtaining the first image data of the section at the first position from the three-dimensional ultrasound volume data according to the inputted orientation of the section.

In one embodiment, before obtaining the prediction path corresponding to the first image data when the adjustment instruction output by the adjustment unit is obtained, the processor 1001 may be further configured to:
search for the prediction path corresponding to the first image data; and
associate the adjustment instruction output by the adjustment unit and the searched prediction path.

In one embodiment, the first image data may include at least one section image data, and the processor 1001 may obtain the prediction path corresponding to the first image data when the adjustment instruction output by the adjustment unit is obtained by:
reconfiguring a correspondence between the adjustment instruction output by the adjustment unit and the prediction path according to a selected one of the at least one section image data; and
obtaining the reconfigured prediction path when the adjustment instruction output by the adjustment section is obtained.

In one embodiment, before obtaining the prediction path corresponding to the first image data when the adjustment instruction output by the adjustment unit is obtained, the processor 1001 may further be configured to:
obtaining the prediction path inputted in a preset manner, or obtaining the prediction path according to an orientation of the first image data; and
reconfiguring a correspondence between the adjustment instruction output by the adjustment unit and the prediction path.

In one embodiment, the processor 1001 may obtain the prediction path inputted in the preset manner, reconfigure a correspondence between the adjustment instruction output by the adjustment unit and the prediction path and adjust the section from the first position to the second position in the three-dimensional ultrasound volume data along the prediction path by:
obtaining the prediction path inputted in the preset manner which is a spatial search path comprising at least two target positions;
reconfiguring a correspondence between the adjustment instruction output by the adjustment unit and the at least two target positions on the spatial search path;
obtaining at least two target positions on the spatial search path when the adjustment instruction output by the adjustment unit is obtained and obtaining at least two prediction paths according to the at least two target positions; and
sequentially adjusting the section from the first position along the at least two prediction paths to multiple second positions in the three-dimensional ultrasound volume data according to the obtained at least two prediction paths.

In one embodiment, the processor 1001 may determine the prediction mode for adjusting the orientation of the section in the three-dimensional ultrasound volume data, obtain the image data from the three-dimensional ultrasound volume data according to the prediction mode and display the obtained image data by:
obtaining a spatial search path, wherein the spatial search path comprises at least two target positions;
obtaining at least two section image data from the three-dimensional ultrasound volume data along the spatial search path; and
displaying the at least two section image data.

In one embodiment, the at least two section image data are tangent or orthogonal to the spatial search path in the three-dimensional ultrasound volume data; or
multiple second image data at the multiple second positions are tangent or orthogonal to the spatial search path in the three-dimensional ultrasound volume data.

In one embodiment, after obtaining the second image data, the processor 1001 may further be configured to:
generate an adjustment display information corresponding to the prediction path, and output the adjustment display information.

In one embodiment, before obtaining the spatial search path, the processor 1001 may further be configured to:
obtain an ultrasound image according to the three-dimensional ultrasound volume data, wherein the ultrasound image comprises at least one of a section image and a three-dimensional image; and
obtain the spatial search path according to an input of an user on the ultrasound image.

Regarding the operations of the processor, reference may be made to the specific implementations of the steps described in connection with FIG. 2 to FIG. 9 and FIG. 18 above, which will not be described again.

In the embodiments of the present disclosure, the three-dimensional ultrasound volume data of the examined target body may be obtained, and the first image data of the section at the first position may be obtained from the three-dimensional ultrasound volume data. Based on the preset prediction path inputted according to the preset manner, the section may be adjusted from the first position to the second position in the three-dimensional ultrasound volume data along the prediction path, and the second image data of the section at the second position in the three-dimensional ultrasound volume data may be obtained and displayed. In addition, the adjustment display information corresponding to the prediction path may be generated, and output. By obtaining the customized prediction path and adjusting the first section at the first position in the three-dimensional ultrasound volume data according to the customized prediction path, the accuracy of the adjustment is increased. By presenting the movement directions during the adjustment through the adjustment display information, the degree of visualization of the adjustment process is increased. In the embodiments of the present disclosure, the three-dimensional ultrasound volume data of the examined target body may be obtained, and the first image data of the section at the first position may be extracted from the three-dimensional ultrasound volume data. Based on the preset prediction path inputted according to the preset manner, the section may be adjusted from the first position to the second position in the three-dimensional ultrasound volume data along the prediction path, and the second image data of the section at the second position in the three-dimensional ultrasound volume data may be obtained and displayed. In addition, the adjustment display information corresponding to the prediction path may be generated, and output. By obtaining the customized prediction path and adjusting the section at the first position in the three-dimensional ultrasound volume data according to the customized prediction path, the accuracy of the adjustment is increased. By presenting the movement directions during the adjustment through the adjustment display information, the degree of visualization of the adjustment process is increased.

A person of ordinary skill in the art may understand that all or part of the processes in the method of the above embodiments can be completed by instructing relevant hardware through a computer program. When executed, it may include the processes of the foregoing method embodiments. Wherein, the storage medium may be a magnetic disk, an optical disk, a read-only memory (Read-Only Memory, ROM) or a random access memory (Random Access Memory, RAM), etc.

The above disclosure is only preferred embodiments of the present invention, and of course it cannot be used to limit the scope of the present invention. Therefore, equivalent changes made according to the claims of the present invention still fall within the scope of the present invention.

1. An image data adjustment method, comprising:
obtaining a three-dimensional ultrasound volume data of an examined target body;
determining a prediction mode for adjusting an orientation of a section in the three-dimensional ultrasound volume data;
obtaining an image data from the three-dimensional ultrasound volume data according to the prediction mode; and
displaying the obtained image data.

2. The method of claim 1, wherein determining the prediction mode for adjusting the orientation of the section in the three-dimensional ultrasound volume data, and obtaining the image data from the three-dimensional ultrasound volume data according to the prediction mode and displaying the obtained image data comprises:
obtaining a first image data of the section at a first position in the three-dimensional ultrasound volume data;
obtaining a prediction path corresponding to the first image data when an adjustment instruction output by an adjustment unit is obtained;
adjusting the section from the first position to a second position in the three-dimensional ultrasound volume data along the prediction path;
obtaining a second image data of the section at the second position in the three-dimensional ultrasound volume data; and
displaying the second image data.

3.-4. (canceled)

5. The method of claim 2, wherein the prediction path comprises any one of an adjustment path of moving a preset distance in one direction and an adjustment path of moving a preset distance in at least two directions.

6. The method of claim 2, wherein obtaining the first image data of the section at the first position in the three-dimensional ultrasound volume data comprises:
obtaining an orientation of the section inputted; and
automatically obtaining the first image data of the section at the first position from the three-dimensional ultrasound volume data according to the inputted orientation of the section.

7. The method of claim 2, before obtaining the prediction path corresponding to the first image data when the adjustment instruction output by the adjustment unit is obtained, further comprising:
searching for the prediction path corresponding to the first image data; and
associating the adjustment instruction output by the adjustment unit and the searched prediction path.

8. The method of claim 2, wherein, the first image data comprises at least one section image data, and obtaining the prediction path corresponding to the first image data when the adjustment instruction output by the adjustment unit is obtained comprises:
reconfiguring a correspondence between the adjustment instruction output by the adjustment unit and the prediction path according to a selected one of the at least one section image data; and
obtaining the reconfigured prediction path when the adjustment instruction output by the adjustment section is obtained.

9. The method of claim 2, wherein obtaining the prediction path when the adjustment instruction output by the adjustment unit is obtained comprises:
obtaining a current position of an indication identifier on a current screen;
obtaining the first image data at the current position; and
obtaining the prediction path corresponding to the first image data at the current position when the adjustment instruction output by the adjustment unit is obtained.

10. The method of claim 2, before obtaining the prediction path corresponding to the first image data when the adjustment instruction output by the adjustment unit is obtained, further comprising:
obtaining the prediction path inputted, or obtaining the prediction path according to an orientation of the first image data; and
reconfiguring a correspondence between the adjustment instruction output by the adjustment unit and the prediction path.

11. The method of claim 2, wherein the adjustment unit is at least one of a virtual adjustment unit and a physical adjustment unit, wherein, the virtual adjustment unit comprises graphical controls arranged on a display interface, and the physical adjustment unit is a hardware having a substantial shape.

12. The method of claim 10, wherein,
the prediction path inputted is a spatial search path comprising at least two target positions;
reconfiguring the correspondence between the adjustment instruction output by the adjustment unit and the prediction path comprises reconfiguring a correspondence between the adjustment instruction output by the adjustment unit and the at least two target positions on the spatial search path; and
obtaining the prediction path corresponding to the first image data when the adjustment instruction output by the adjustment unit is obtained comprises:
obtaining at least two target positions on the spatial search path when the adjustment instruction output by the adjustment unit is obtained and obtaining at least two prediction paths according to the at least two target positions.

13. The method of claim 12, wherein adjusting the section from the first position to the second position in the three-dimensional ultrasound volume data along the prediction path comprises:
sequentially adjusting the section from the first position along the at least two prediction paths to multiple second positions in the three-dimensional ultrasound volume data according to the obtained at least two prediction paths.

14. The method of claim 1, wherein determining the prediction mode for adjusting the orientation of the section in the three-dimensional ultrasound volume data, and obtaining the image data from the three-dimensional ultrasound volume data according to the prediction mode and displaying the obtained image data comprises:
obtaining a spatial search path, wherein the spatial search path comprises at least two target positions;
obtaining at least two section image data from the three-dimensional ultrasound volume data along the spatial search path; and
displaying the at least two section image data.

15. The method of claim 14, wherein, the at least two section image data are tangent or orthogonal to the spatial search path in the three-dimensional ultrasound volume data.

16. The method of claim 2, after obtaining the second image data, the method further comprising:
generating an adjustment display information corresponding to the prediction path, and outputting the adjustment display information.

17. The method of claim 14, wherein the spatial search path is obtained by a drawing of an user on an image.

18. The method of claim 14, before obtaining the spatial search path, further comprising:
obtaining an ultrasound image according to the three-dimensional ultrasound volume data, wherein the ultrasound image comprises at least one of a section image and a three-dimensional image; and
obtaining the spatial search path according to an input of an user on the ultrasound image.

19.-21. (canceled)

22. An ultrasound imaging device, comprising an ultrasound probe, a transmitting and receiving circuit, an image processing unit, a human-computer interaction device, a display, a memory and a processor, wherein,
the ultrasound probe is configured to transmit ultrasonic waves to a target body;
the transmitting and the receiving circuit is configured to excite the ultrasound probe to transmit an ultrasonic beam to the target body and receive echoes of the ultrasonic beam to obtain ultrasound echo signals;
the image processing unit is configured to obtain a three-dimensional ultrasound volume data according to the ultrasound echo signals;
the human-computer interaction device is configured to obtain an instruction inputted by an user;
the memory is configured to store a computer program;
the processor is configured to execute the computer program which, when executed by the processor, lead the processor to:
determine a prediction mode for adjusting an orientation of a section in the three-dimensional ultrasound volume data;
obtain an image data from the three-dimensional ultrasound volume data according to the prediction mode; and
display the obtained image data.

23. The device of claim 22, wherein the processor determines the prediction mode for adjusting the orientation of the section in the three-dimensional ultrasound volume data, and obtains the image data from the three-dimensional ultrasound volume data according to the prediction mode and displays the obtained image data by:
obtaining a first image data of the section at a first position in the three-dimensional ultrasound volume data;
obtaining a prediction path corresponding to the first image data when an adjustment instruction inputted is obtained through the human-computer interaction device;
adjusting the section from the first position to a second position in the three-dimensional ultrasound volume data along the prediction path;
obtaining a second image data of the section at the second position in the three-dimensional ultrasound volume data; and
displaying the second image data on the display.

24.-33. (canceled)

34. The device of claim 22, wherein the processor determines the prediction mode for adjusting the orientation of the section in the three-dimensional ultrasound volume data, and obtains the image data from the three-dimensional ultrasound volume data according to the prediction mode and displays the obtained image data by:
obtaining a spatial search path, wherein the spatial search path comprises at least two target positions;
obtaining at least two section image data from the three-dimensional ultrasound volume data along the spatial search path; and
displaying the at least two section image data.

35.-37. (canceled)

38. The device of claim 34, wherein, before obtaining the spatial search path, the processor is further configured to:
obtain an ultrasound image according to the three-dimensional ultrasound volume data, wherein the ultrasound image comprises at least one of a section image and a three-dimensional image; and
obtain the spatial search path according to an input of an user on the ultrasound image.
